(19) United States
(12) Patent Application Publication
(10) Pub. No.: US 2006/0142315 A1
(43) Pub. Date: Jun. 29, 2006

(54) Substituted biaryl piperazinyl-pyridine analogues

(76) Inventors: Andre Rosowsky, Needham, MA (US); Ronald A. Forsch, Las Vegas, NV (US)

Correspondence Address: EDWARDS & ANGELL, LLP, P.O. BOX 55874, BOSTON, MA, 02205, US

(21) Appl. No.: 11/227,783

(22) Filed: Sep. 14, 2005

Related U.S. Application Data
(63) Continuation of application No. PCT/US04/07753, filed on Mar. 12, 2004.
(60) Provisional application No. 60/454,575, filed on Mar. 14, 2003.

(51) Int. Cl. A61K 31/517; A61K 31/505

(52) U.S. Cl. 514/266.4; 544/291; 544/323; 514/275

(57) ABSTRACT

The present invention relates to dihydrofolate reductase inhibitors having an aromatic group and a heteroaromatic group linked by a methylene group; and methods of treatment and pharmaceutical compositions that utilize or comprise one or more of such dihydrofolate reductase inhibitors. More particularly, the present invention relates to dihydrofolate reductase inhibitors having a substituted aromatic group and a heteroaromatic group linked by a methylene group wherein at least one of the aromatic group substituents is a lipophilic residue comprising at least one acidic functional group.

This application claims the benefit of U.S. Provisional Patent Application 60/454,575, filed Mar. 14, 2003, which application is incorporated by reference.

This work was supported by research grant RO1-AI29904 from the National Institute of Allergy and Infectious Diseases (NIAID), U.S. Department of Health and Human Services. The government has certain rights in the invention.

### BACKGROUND OF THE INVENTION

1. Field of the Invention

The present invention provides new compounds which are active dihydrofolate reductase (DHFR) inhibitors, more particularly, the compounds of the invention selectively inhibit activity of DHFR enzymes from parasitic organisms such as Pneumocystis carinii (Pc), Toxoplasma gondii (Tg), and Mycobacterium avium (Ma). The present Invention further provides therapeutic methods of treating patients suffering from or susceptible to parasitic infections.

2. Background

Piritrexim (A), a lipophilic inhibitor of the key metabolic enzyme dihydrofolate reductase (DHFR) has been studied intensively as an anticancer drug, and more recently was identified as a potent inhibitor of the enzyme from Pneumocystis carinii (Pc) and Toxoplasma gondii (Tg), two opportunistic parasites known to be potentially life-threatening in patients with acquired immunodeficiency syndrome (AIDS). See, for example, Grivsky, E. M.; Lee, S.; Sigel, C. W.; Duch, D. S.; Nichol, C. A. J. Med Chem. 1980; 23, 327-329;Sigel, C. W.; Macklin, A. W.; Woolley, J. L., Jr.; Johnson N. W.; Collier, M. A.; Blum, M. R.; Clendeninn, N. J.; Everitt, J. M.; Grebe, G.; Mackars, A.; Foss, R. G.; Duch, D. S.; Bowers, S. W.; Nichol, C. A. NCI Monogr. 1987; 5, 111-120; Laszlo, J.; Brenckman, W. D., Jr.; 1987; 5, 121-125; and Kovacs, J.; Allegra, C. A.; Swan, J. C.; Drake, J. C.; Parrillo, J. E.; Chabner, B. A.; Masur, H. Antimicrob. Agents Chemother. 1988; 32, 430-433. A notable structural feature of (A)is the short CH₂ bridge between the two halves of the molecule. This bridge is also present in trimethoprim (B), another lipophilic DHFR inhibitor widely used to for anti-Pc and anti-Tg prophylaxis and therapy in AIDS patients, usually in combination with a sulfa drug to enhance efficacy. For an excellent historical account of the chemical and pharmaceutical development of the older lipophilic DHFR inhibitors pyrimethamine and trimethoprim, see: Roth, B.; Cheng, C. C. Progr. Med Chem. 1982; 19, 269-331; Fischl, M. A.; Dickinson, G. M.; La Voie, L. J. Am. Med. Assoc. 1988; 259, 1185-1189; and Medina, I.; Mills, J.; Leoung, G.; Hopewell, P. C.; Lee, B.; Modin, G.; Benowitz, N.; Wofsy, C. B. N. Engl. J. Med 1990; 323, 776-782. Two other members of this class that have been used clinically against these infections are pyrimethamine (C), in which the two halves of the molecule are linked without a CH₂ bridge, and trimetrexate (D), which contains a longer CH₂NH bridge. In addition to the fact that it contains a longer bridge, (D) differs from (A) in being a quinazoline as opposed to a pyrido[2,3-d]pyrimidine. See for example, Bertino, J. R.; Sawicki, W. L.; Moroson, B. A.; Cashmore, A. R.; Elslager, E. F. Biochem. Pharmacol. 1979; 28, 1983-1987; Elslager, E. F.; Johnson, E. L.; Werbel, L. M. J. Med Chem. 1983; 26, 1753-1760; and Sattler, F. R.; Frame, P.; Davis, R.; Nichols, L.; Shelton, B.; Akil, B.; Baugman, R.; Hughlett, C.; Weiss, W.; Boylen, C. T.; van der Horst, C.; Black, J.; Masur, H.; Feinberg, J. J. Infect. Dis. 1994; 170, 165-172.

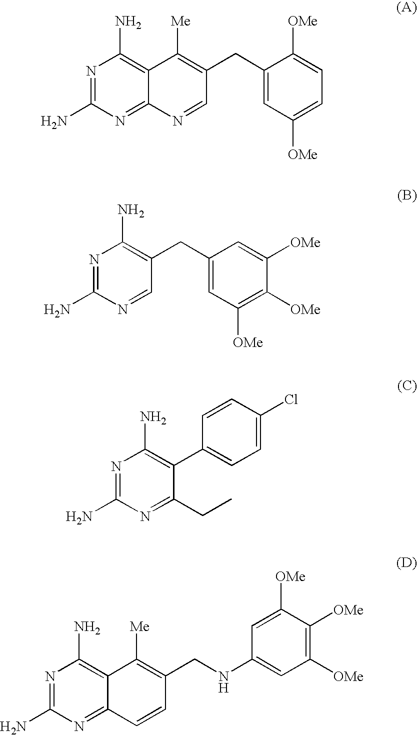

There has been reported examples of certain lipophilic DHFR inhibitors in which the fused 2,4-diaminopyrimidine ring system and the aryl side chain are separated by a short O or S bridge, as in (E) and (F). The only quinazoline antifolates reported to date, however, are those in which the bridge is CH₂ are the 5,6,7,8-tetrahydro derivatives (G). See for example Elsiager, E. F.; Clarke, J.; Johnson, J.; Werbel, L. M. Davoll, J. J. Heterocycl. Chem. 1972; 9, 759-773; Hynes, J. B.; Ashton, W. T.; Merriman, H. G., III, Walker, F. C., III. J. Med Chem. 1974; 17, 682-684; and Rosowsky A; Papoulis, A. T.; Forsch, R. A.; Queener, S. F. J. Med Chem. 1999; 42, 1007-1017.

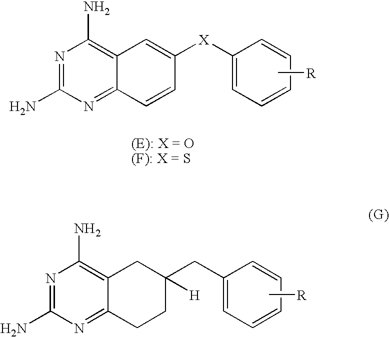

International application PCT/US02/37155, filed Nov. 19, 2002, to Rosowsky et al recites a related class of compounds having a heterocylic ring structure and a phenyl group linked by a methylene group where the phenyl or naphthyl group may be substituted with a variety of substituents.

It would be desirable to have new methods for synthesis of biologically active DHFR inhibitors such as compounds (A)-(G) and the like.

### SUMMARY OF THE INVENTION

The present invention provides a new class of compounds possessing selective binding affinities to parasitic dihydrofolate reductase enzymes (DHFR). More particularly, the compounds of the invention selectively bind to at least one parasitic DHFR enzyme with greater binding affinity than mammalian DHFR enzymes such as DHFR from rat, primates or human. Compounds of the invention typically possess at least one lipophilic, e.g., hydrophobic, substituent which is functionalized with an acidic residue. The acid functionalized hydrophobic substituent is suitable for binding to a hydrophobic channel present in mammalian and parasitic DHFR enzymes. Although not wishing to be bound by theory, it is believed that a basic residue present in the hydrophobic channel of parasitic DHFR enzymes results in the compounds of the invention which possess an acid functionalized hydrophobic substituent having a higher affinity for parasitic DHFR enzymes relative to other DHFR enzymes including mammalian DHFR enzymes.

The present invention provides compounds of the formula: 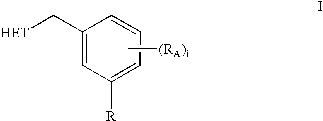
wherein

HET is selected from

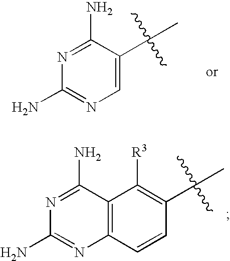

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination from a group of the formula: 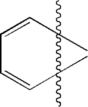
which may be optionally substituted;

R is L-(CR¹R²)ₙ—C(O)—X or a group of the formula:

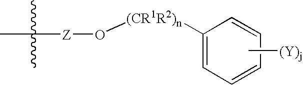

L is a single bond, an optionally substituted 1,2-vinylidene group, or a 1,2 acetylene-diyl group;

X is selected from OH, SH, NH₂, optionally substituted alkoxy, optionally substituted benzyloxy, or optionally substituted aryloxy and salts thereof; and

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

i is an integer from 0 to about 4;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

Preferred compounds of Formula I are DHFR inhibitors and more preferably selectively inhibit DHFR activity in parasitic organisms. Thus preferred compounds of the invention are capable of selectively inhibiting DHFR in parasitic organisms with minimal or no disruption of DHFR activity in the host organism.

The present invention further provides pharmaceutical compositions comprising a pharmaceutically acceptable carrier and at least one compound according to Formula I or any subformula thereof.

Methods of treating a patient, e.g., a mammalian patient, who is suffering from or is susceptible to a parasitic invention are also provided by the present invention. Typically a pharmaceutically effective dose of at least one compound according to Formula I or a subformula thereof is administered to the patient in need to prevent or ameliorate the effects of a parasitic infection.

Other aspects of the of the invention are discussed infra.

### DETAILED DESCRIPTION OF THE INVENTION

Preferred compounds of the invention include those compounds according to one of Formula II or Formula III: 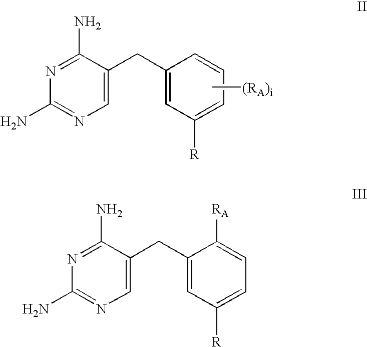
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination form a group of the formula: 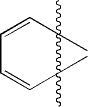
which may be optionally substituted;

R is L-(CR¹R²)ₙ—C(O)—X or a group of the formula:

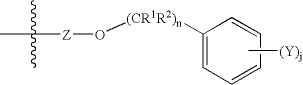

L is a single bond, an optionally substituted 1,2-vinylidene group, or a 1,2 acetylene-diyl group;

X is selected from OH, SH, NH₂, optionally substituted alkoxy, optionally substituted benzyloxy, or optionally substituted aryloxy and salts thereof; and

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

i is an integer from 0 to about 4;

j is an integer from 0 to about 5; and

R¹ and R² are independently selected at each occurrence from the group consisting of hydrogen, methyl, ethyl, chloro, fluoro, hydroxy, amino, trifluoromethoxy, or methoxy.

Preferred compounds according to Formula II include those compounds represented by Formula IV:

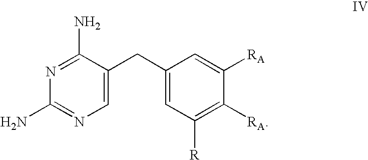

In certain preferred compounds of Formula IV, each occurrence of R_{A} is an optionally substituted alkoxy group, or more preferably, each R_{A} is a methoxy residue.

The invention further provides compounds according to Formula V and according to Formula VI: 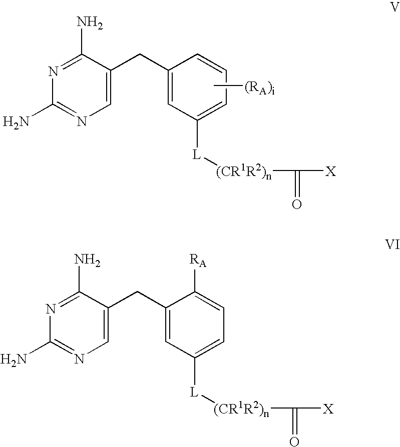
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination form a group of the formula: 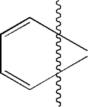
which may be optionally substituted;

L is a single bond or a 1,2 acetylene-diyl group;

X is selected from OH, SH, NH₂, optionally substituted alkoxy, optionally substituted benzyloxy, or optionally substituted aryloxy and salts thereof; and

n is an integer of from 1 to about 12; or

i is an integer from 0 to about 4;

j is an integer from 0 to about 5; and

R¹ and R² are independently selected at each occurrence from the group consisting of hydrogen, methyl, ethyl, chloro, fluoro, hydroxy, amino, trifluoromethoxy, or methoxy.

In preferred compounds according to any one of Formula II, III, IV, V, and VI, the L group is either a single bond or a 1,2-acetylenediyl group, i.e., a C—C triple bond (—C≡C—).

Yet other preferred compounds according to Formula III include those compounds represented by the structure:

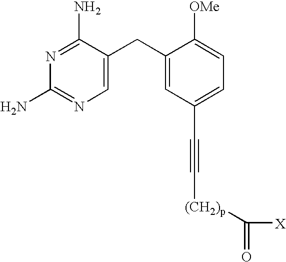

Still other preferred compounds according to Formula II and Formula IV include those compounds represented by the structure: 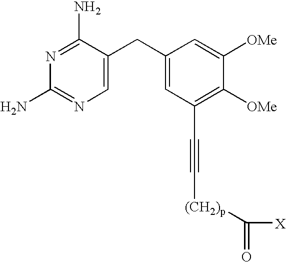
wherein p is an integer of from 1 to 12 or from about 1 to about 10, or more preferably of from about 2 to about 8. In certain particularly preferred compounds, p is 2, 3, or 4.

Other preferred compounds according to Formula III include those compounds represented by the structure: 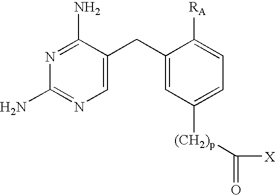
wherein p is an integer from 3 to about 14.

Other preferred compounds of Formula II which are provided by the invention include those compounds represented by one of Formula VII, Formula VIII or Formula IX: 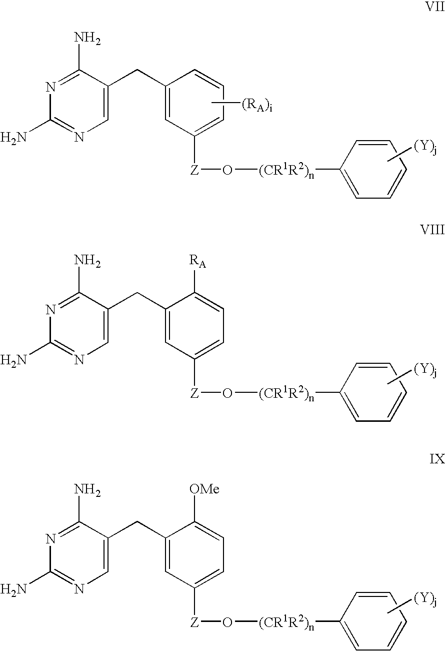
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 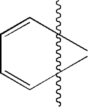
   which may be optionally substituted;

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

i is an integer from 0 to about 4;

j is an integer from 0 to about 5; and

R¹ and R² are independently selected at each occurrence from the group consisting of hydrogen, methyl, ethyl, chloro, fluoro, hydroxy, amino, trifluoromethoxy, or methoxy.

Preferred compounds according to Formula IX include those compounds in which:

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

j is an integer from 0 to about 5; and

R¹ and R² are independently selected at each occurrence from the group consisting of hydrogen, methyl, ethyl, chloro, fluoro, hydroxy, amino, trifluoromethoxy, or methoxy.

Other particularly preferred compounds according to Formula IX include compounds according to the formula: 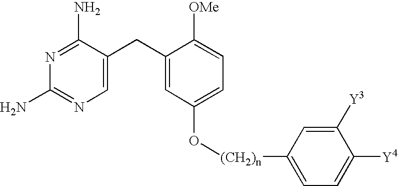
wherein at least one Y³ and Y⁴ is a carboxylic acid residue or a salt thereof.

Other preferred compounds according to Formula II include those compounds according to Formula X: 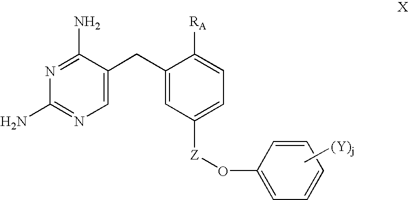
wherein

R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a hydrocarbon chain having from 1 to about 6 carbon atoms in the chain and between 0 and about 2 double or triple bonds in the chain;

j is an integer from 0 to about 5; and

R¹ and R² are independently selected at each occurrence from the group consisting of hydrogen, methyl, ethyl, chloro, fluoro, hydroxy, amino, trifluoromethoxy, or methoxy.

Preferred compounds of Formula X provided by the invention include those compounds according to the formula: 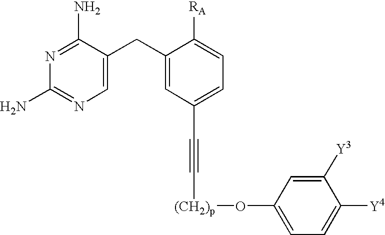
wherein

at least one Y³ and Y⁴ is a carboxylic acid residue or a salt thereof; and

p is an integer of from 1 to about 4.

Still other preferred compounds of Formula X provided by the invention include those compounds according to the formula: 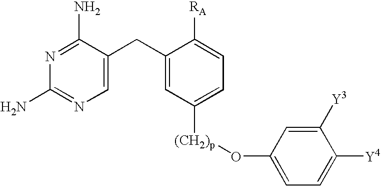
wherein

at least one Y³ and Y⁴ is a carboxylic acid residue or a salt thereof; and

p is an integer of from 3 to about 6.

Other preferred compounds of the invention include those compounds according to any one of Formula XI, XII, or XIII: 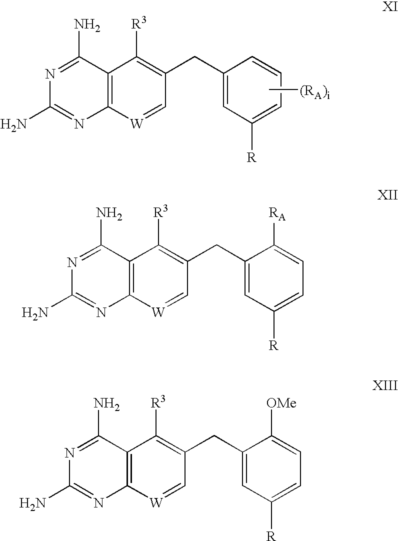
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination form a group of the formula: 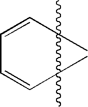
which may be optionally substituted;

R is L-(CR¹R²)ₙ—C(O)—X or a group of the formula:

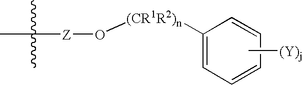

L is a single bond, an optionally substituted 1,2-vinylidene group, or a 1,2 acetylene-diyl group;

W is a CH group or N;

X is selected from OH, SH, NH₂, optionally substituted alkoxy, optionally substituted benzyloxy, or optionally substituted aryloxy and salts thereof; and

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

i is an integer from 0 to about 4;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

Preferred compounds according to Formula XI provided by the present invention include those compounds according to any one of Formula XIV or XV: 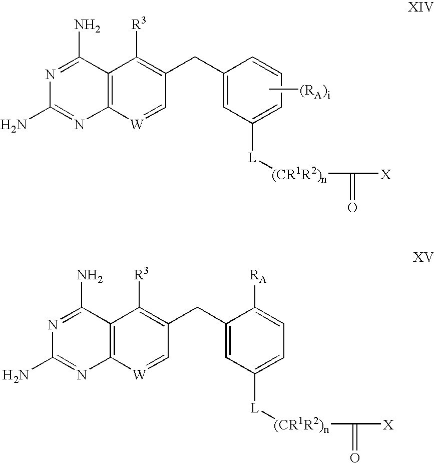
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination form a group of the formula: 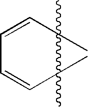
which may be optionally substituted;

L is a single bond or a 1,2 acetylene-diyl group;

W is a CH group or N;

X is selected from OH, SH, NH₂, optionally substituted alkoxy, optionally substituted benzyloxy, or optionally substituted aryloxy and salts thereof; and

n is an integer of from 1 to about 12; or

i is an integer from 0 to about 4;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

In preferred compounds according to any one of Formula XII, XIII, XIV or XV, the L group is either a single bond or a 1,2-acetylenediyl group, i.e., a C—C triple bond (—C≡C—).

Particularly preferred compounds of the invention according to Formula XV include those compounds of the formula: 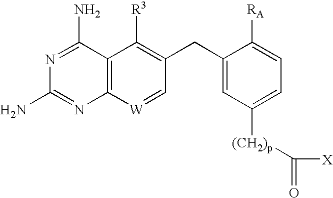
wherein p is an integer from 3 to about 14.

Still other particularly preferred compounds of the invention according to Formula XV include those compounds of the formula: 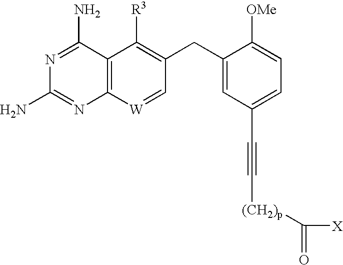
wherein p is an integer from 1 to about 12.

The invention further provides compounds according to Formula XI according to any one of Formula XVI, XVII, or XVIII: 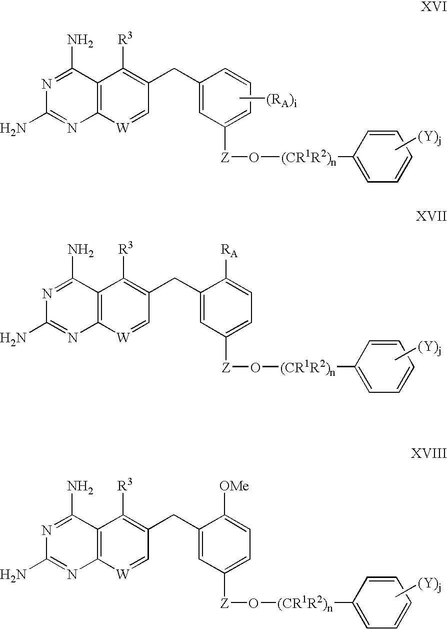
wherein

R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or

two adjacent R_{A} groups taken in combination form a group of the formula: 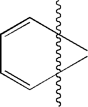
which may be optionally substituted;

W is a CH group or N;

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

i is an integer from 0 to about 4;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

Particularly preferred compounds according to Formula XVIII include those compounds where:

W is a CH group or N;

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a single bond or hydrocarbon chain having from 1 to about 6 carbon atoms in the chain and between 0 and about 2 double or triple bonds in the chain;

n is an integer of from 0 to about 12, wherein the sum of n and the number of carbon atoms in the hydrocarbon chain of the Z group is greater than 0;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

Particularly preferred compounds according to Formula XVIII, which are provided by the invention, include compounds according to formula: 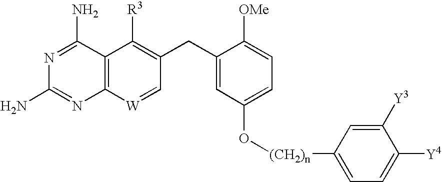
wherein

at least one Y³ and Y⁴ is a carboxylic acid residue or a salt thereof.

In yet another embodiment, the invention provides compounds according to Formula XIX: 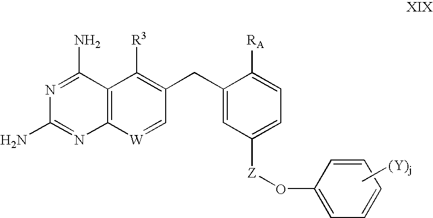
wherein

R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;

W is a CH group or N;

Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;

Z is a hydrocarbon chain having from 1 to about 6 carbon atoms in the chain and between 0 and about 2 double or triple bonds in the chain;

j is an integer from 0 to about 5; and

R¹, R², R³ are independently selected at each occurrence from the group consisting of hydrogen, methyl, ethyl, chloro, fluoro, hydroxy, amino, trifluoromethoxy, or methoxy.

Particularly preferred compounds according to Formula XIX, which are provided by the invention, include compounds according to formula: 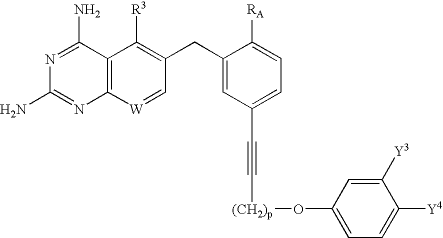
wherein

at least one Y³ and Y⁴ is a carboxylic acid residue or a salt thereof; and

p is an integer of from 1 to about 4.

The invention provides other particularly preferred compounds according to Formula XIX, including compounds according to formula: 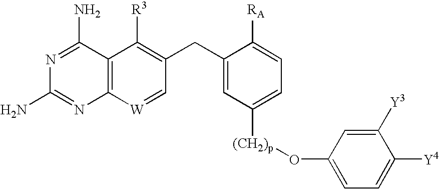
wherein

at least one Y³ and Y⁴ is a carboxylic acid residue or a salt thereof; and

p is an integer of from 3 to about 6.

Non-limiting examples of preferred compounds include compounds of Formula I selected from the group consisting of:

2,4-Diamino-5-[5′-(4-carboxy-1-butynyl)-2′-methoxy)benzyl]pyrimidine (Compound 2);

2,4-Diamino-5-[5′-(4-carboxy-1-pentynyl)-2′-methoxy)benzyl]pyrimidine (Compound 3);

2,4-Diamino-5-[5′-(6-carboxy-1-hexynyl)-2′-methoxy)benzyl]pyrimidine (Compound 4);

2,4-Diamino-5-[5′-(4-carboxybutyl)-2′-methoxy)benzyl]pyrimidine (Compound 5);

2,4-Diamino-5-[5′-(5 -carboxypentyl)-2′-methoxy)benzyl]pyrimidine (Compound 6);

2,4-Diamino-5-[5′-(6-carboxyhexyl)-2′-methoxy)benzyl]pyrimidine (Compound 7);

2,4-Diamino-5-[2′-methoxy-5′-(3″-carboxybenzyloxy)benzylpyrimidine (Compound 8);

2,4-Diamino-5-(2′-methoxy-5′-(4″-carboxybenzyloxy)benzylpyrimridine (Compound 9);

2,4-Diamino-5-[2′-methoxy-5′-[3-(2″-carboxyphenoxy)propyn-1-yl]benzyl]pyrimidine (Compound 10);

2,4-Diamino-5-[2′-methoxy-5′-[3 -(3″-carboxyphenoxy)-1-propynyl]benzyl]pyrimidine (Compound 11); and

6-[5-(2,4-Diamino-pyrimidin-5-ylmethyl)-2,3-dimethoxy-phenyl]-hex-5-ynoic acid (Compound 49).

Particularly preferred compounds according to any one of Formula I through XIX including any subformulae thereof which are provided by the present invention are lipophilic inhibitors of dihydrofolate reductase. More preferably, the compounds of the invention are selective inhibitors of parasitic dihydrofolate reductase including, but not limited to, dihydrofolate reductase of one or more parasites selected from a Pneumocystis carinii (Pc), Toxoplasma gondii (Tg), or a Mycobacterium avium (Ma).

Suitable halogen substituent groups or halide groups of compounds of the invention, including compounds of any one of Formula I through XIX and any subformulae thereof as defined above, include F, Cl, Br and I. Alkyl groups of compounds of the invention preferably have from 1 to about 12 carbon atoms, more preferably 1 to about 8 carbon atoms, still more preferably 1 to about 6 carbon atoms. As used herein, the term alkyl unless otherwise modified refers to both cyclic and noncyclic groups, although of course cyclic groups will comprise at least three carbon ring members. Straight or branched chain noncyclic alkyl groups are generally more preferred than cyclic groups, particularly branched chain groups such as isopropyl and t-butyl. Preferred alkenyl groups of compounds of the invention have one or more unsaturated linkages and from 2 to about 12 carbon atoms, more preferably 2 to about 8 carbon atoms, still more preferably 2 to about 6 carbon atoms. The term alkenyl as used herein refer to both cyclic and noncyclic groups, although straight or branched noncyclic groups are generally more preferred, particularly branched chain groups. Preferred alkoxy groups of compounds of the invention include groups having one or more oxygen linkages and from 1 to about 12 carbon atoms, more preferably from 1 to about 8 carbon atoms, and still more preferably 1 to about 6 carbon atoms. Preferred thioalkyl groups of compounds of the invention include those groups having one or more thioether linkages and from 1 to about 12 carbon atoms, more preferably from 1 to about 8 carbon atoms, and still more preferably 1 to about 6 carbon atoms. Preferred aminoalkyl groups include those groups having one or more primary, secondary and/or tertiary amine groups, and from 1 to about 12 carbon atoms, more preferably 1 to about 8 carbon atoms, still more preferably 1 to about 6 carbon atoms. Substituted and unsubstituted mono and dialkylamino groups are particularly preferred, especially where each alkyl chain of the group has from 1 to about 6 carbon atoms. Preferred alkylsulfoxide of compounds of the invention have one or more sulfoxide groups, more typically one sulfoxide group, and from 1 to about 12 carbon atoms, more preferably 1 to about 8 carbon atoms, still more preferably 1 to about 6 carbon atoms. Preferred sulfonoalkyl groups of compounds of the invention have one or more sulfono (SO₂) groups, more typically one or two sulfono groups, and from 1 to about 12 carbon atoms, more preferably 1 to about 8 carbon atoms, still more preferably 1 to about 6 carbon atoms. Preferred alkanoyl groups of compounds of the invention include groups having one or more carbonyl groups, more typically one or two carbonyl groups, and from 1 to about 12 carbon atoms, more preferably 1 to about 8 carbon atoms, still more preferably 1 to about 6 carbon atoms. Preferred alkylcarboxyamino groups include those groups of the formula —NHCOOR where R is substituted or unsubstituted alkyl having from 1 to about 10 carbon atoms, more preferably 1 to about 6 carbon atoms. Suitable heteroaromatic groups of compounds of the invention contain one or more N, O or S atoms and include, e.g., quinolinyl, pyridyl, pyrazinyl, indolyl, carbazoyl, luryl, pyrrolyl, thienyl, thiazolyl, aminothioazole such as 2-aminothiazole, pyrazole, oxazolyl, imidazolyl, indolyl, benzofuranyl, benzothiazol and pyridonal including 2-pyridonals and 4-pyridonals, particularly pyridonal substituted at one or more ring positions by moieties such as hydroxy, alkanoyl such as acetate, alkylaminocarbonyl having from 1 to about 8 carbon atoms and alkoxycarbonyl having from 1 to about 8 carbon atoms. Suitable heteroalicyclic groups of compounds contain one or more N, O or S atoms and include, e.g., aziridinyl, azetidinyl, tetrahydrofuranyl, tetrahydropyranyl, pyrrolidino, 1,2,3,6-tetrahydropyridino, piperazino, piperidinyl, morpholino and thiomorpholino.

Substituted moieties of compounds of the invention, including substituted R_{A}, R¹, R², R³, W, X, Y, and Z groups, may be substituted at one or more available positions by one or more suitable groups such as, e.g., halogen such as fluoro, chloro, bromo and iodo; cyano; hydroxyl; nitro; alkyl groups including those groups having 1 to about 12 carbon atoms or from 1 to about 6 carbon atoms, preferably noncyclic alkyl groups including branched chain groups such as isopropyl and t-butyl; alkenyl and alkynyl groups including groups having one or more unsaturated linkages and from 2 to about 12 carbon or from 2 to about 6 carbon atoms; alkylthio groups including those moieties having one or more thioether linkages and from 1 to about 12 carbon atoms or from I to about 6 carbon atoms; and, in at least preferred aspects of the invention, alkoxy groups having those having one or more oxygen linkages and from 1 to about 12 carbon atoms or 1 to about 6 carbon atoms; and aminoalkyl groups such as groups having one or more N atoms (which can be present as primary, secondary and/or tertiary N groups) and from 1 to about 12 carbon atoms or from 1 to about 6 carbon atoms.

The present invention provides a straightforward and versatile approach that can lend itself to the synthesis of a rich library of previously unknown biologically active DHFR inhibitors in which a substituted aryl group is linked directly to the 2,4-diamino heterocyclic moiety via a CH₂ bridge. The present invention also provides a new class of biologically active compounds according to formula I which are active DHFR inhibitors.

Thus the compounds of the present invention, particularly compounds of any one of Formula I through XIX and any subformulae thereof are useful as pharmaceuticals for the treatment of mammals, including humans, particularly for the treatment of mammals having immunodeficiency disorder and/or HIV positive, particularly a human suffering from or susceptible to AIDS. Compounds of the invention typically combat parasitic infections which are known to inflict HIV-positive or patients suffering from or susceptible to AIDS. Thus, the invention provides a method for the treatment of AIDS, in mammals including humans, the method comprising administration of an effective amount of one or more compounds of the invention in a pharmaceutically useful form, once or several times a day or other appropriate schedule, orally, rectally, parenterally (particularly intravenously), topically, etc.

For such treatment, the compounds of the invention are administered in effective amounts and in appropriate dosage form ultimately at the discretion of the medical or veterinary practitioner. For example, as known to those skilled in the art, the amount of compounds of the invention required to be pharmaceutically effective will vary with a number of factors such as the mammal's weight, age and general health, the efficacy of the particular compound and formulation, route of administration, nature and extent of the condition being treated, and the effect desired. The total daily dose may be given as a single dose, multiple doses, or intravenously for a selected period. Efficacy and suitable dosage of a particular compound can be determined by known methods including through use of the protocols of Example 14 which follows. More particularly, for treatment of a tumor in a mammal such as a human, particularly when using more potent compounds of the invention, a suitable effective dose of the compound of the invention according to Formula I through XIX or a subformula thereof will be in the range of 0.01 to 100 milligrams per kilogram body weight of recipient per day, preferably in the range of 1 to 10 milligrams per kilogram body weight of recipient per day. The desired dose is suitably administered once daily, or as several sub-doses, e.g. 2 to 4 sub-doses administered at appropriate intervals through the day, or other appropriate schedule. Such sub-doses may be administered as unit dosage forms, e.g., containing from 0.2 to 200 milligrams of compound(s) of the invention per unit dosage, preferably from 2 to 20 milligrams per unit dosage.

The compounds of the present invention may be suitably administered to a subject as a pharmaceutically acceptable salt. Such salts can be prepared in a number of ways. For example, where the compound comprises a basic group such as an amino group, salts can be formed from an organic or inorganic acid, e.g. hydrochloride, sulfate, hemisulfate, phosphate, nitrate, acetate, oxalate, citrate, maleate, etc.

The therapeutic compound(s) may be administered alone, or as part of a pharmaceutical composition, comprising at least one compound of the invention together with one or more acceptable carriers thereof and optionally other therapeutic ingredients, e.g., other AIDS agents or part of a cocktail of therapeutic agents. The carrier(s) must be “acceptable” in the sense of being compatible with the other ingredients of the formulation and not deleterious to the recipient thereof.

The compositions include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well known in the art of pharmacy.

Such methods include the step of bringing into association the to be administered ingredients with the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions suitable for topical administration include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Compositions suitable for topical administration to the skin may be presented as ointments, creams, gets and pastes comprising one or more compounds of the present invention and a pharmaceutically acceptable carrier. A suitable topical delivery system is a transdermal patch containing the ingredient to be administered.

Compositions suitable for rectal administration may be presented as a suppository with a suitable base comprising, for example, cocoa butter or a salicylate.

Compositions suitable for nasal administration wherein the carrier is a solid include a coarse powder having a particle size, for example, in the range 20 to 500 microns which is administered in the manner in which snuff is taken, i.e., by rapid inhalation through the nasal passage from a container of the powder held close up to the nose. Suitable formulations wherein the carrier is a liquid, for administration, as for example, a nasal spray or as nasal drops, include aqueous or oily solutions of the active ingredient.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above the formulations of this invention may include other agents conventional in the art having regard to the type of formulation in question, for example, those suitable for oral administration may include flavoring agents.

Chemistry

As shown in Scheme 1, the well-known Pd-catalyzed Sonogashira reaction between an aryl halide and a terminal alkyne provided convenient access to the analogs of 1a and 1b with a 5′-(ω-carboxy-1-alkynyl) side chain or a 5′-(ω-carboxyalkyl) side chain. See, for example, Sonogashira, K; Tohda, Y.; Hagihara, B. Tetrahedron Lett. 1975, 4467-4470; and Takahashi, S.; Kuroyama, Y.; Sonogashira, K.; Hagihara, N. Synthesis 1980, 627-630. The key intermediates needed for the coupling reaction were the terminal acetylenic esters 15-17 and the iodide 19, the latter of which was obtained from 2,4-diamino-5-(2′-methoxybenzyl)pyrimidine (18) by reaction with iodine monochloride as described by Calas and coworkers (Calas, M.; Barbier, A.; Giral, L.; Balmayer, B.; Despaux, E. Eur. J. Med. Chem.—Chim. Ther. 1982, 17, 497-504). The esters were readily prepared from the Cs salts of the commercially available acids 12-14 with benzyl bromide in dry DMF. Without purification, 15-17 were condensed with 19 in the presence of Et₃N and catalytic amounts of (Ph₃P)₂PdCl₂ and CuI in dry DMF at 60° C. under N₂. Because of their limited solubility in nonpolar solvents, the resulting acetylenic esters 20-22 did not lend themselves easily to column chromatography on silica gel, and therefore were converted directly to the acids 2-4 by brief treatment with NaOH in DMSO at room temperature. The yield of 2 and 4 after purification by preparative HPLC on C₁₈ silica gel using an isocratic mixture of MeCN and 0.1 MNH₄OAc, pH 7.4, as the eluent was 32% and 36%, respectively. In the case of 3, we did not use HPLC but instead attempted to recrystallize the compound from MeOH. This proved to be a bad choice, inasmuch as heating the acetylenic acid in MeOH led to a sharp drop in final purified yield because of extensive degradation to a resinous, base-insoluble product of unknown composition (the crude yield had been comparable to that of 2 and 4).

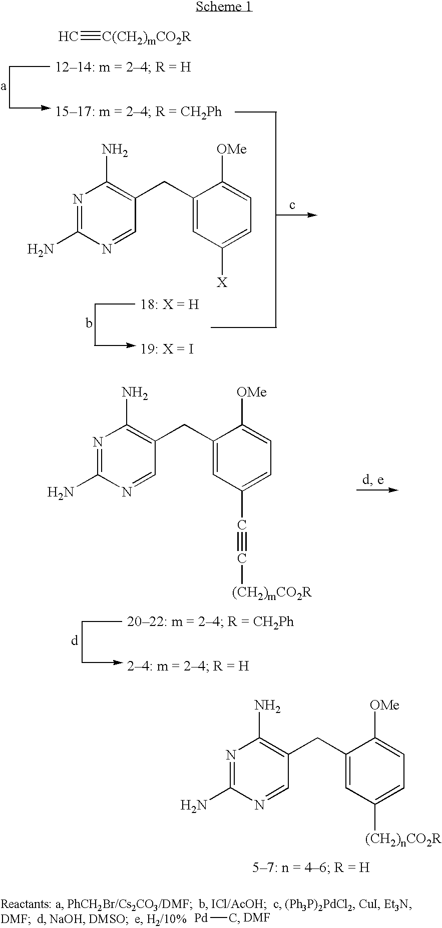

We had originally chosen benzyl esters in Scheme 1 with the intent of using catalytic hydrogenation (H₂/Pd—C) to reductively cleave the benzyl group and simultaneously reduce the triple bond to form the saturated acids 5-7, respectively. However, when hydrogenation was attempted with 21 in DMF solution, the triple bond was reduced but the ester group survived. When reduction was performed in a 1:1 mixture of DMF and glacial AcOH with a view to protonating the diaminopyrimidine moiety, which we suspected might be inactivating the Pd—C catalyst enough to prevent debenzylation, reduction occurred; however the product proved to be totally insoluble in base, and thus could not be a carboxylic acid. Fortunately, conversion of the acetylenic esters to the desired saturated acids was achieved satisfactorily by first cleaving the ester group with NaOH and hydrogenating the Na salt in situ in aqueous solution, or in the case of 7 by isolating the free acid and carrying out the reduction in DMF solution. In contrast to 3 (see above), purification of 5 was achieved satisfactorily by recrystallization from MeOH. Preparative HPLC (C₁₈ silica gel, 20% MeCN in 0.1 M NH₄OAc, pH 7.4) was used in the case of 6; however, analytically pure 7 could be obtained by simply dissolving the product of the hydrogenation reaction in dilute NaOH and acidifying the solution with AcOH. The overall two-step yields of the purified acids 5-7 was 35%, 46%, and 77%, respectively. The ¹H NMR spectra of all three compounds were consistent with reduction of the C≡C bond, in that there were four additional CH₂ hydrogens and a slight upfield shift for the aromatic protons ortho to the ω-carboxyalkyl side chain.

Although we had originally planned to prepare compounds 8 and 9 from the Na salt of 2,4-diamino-5-(2′-methoxy-4′-hydroxybenzyl)pyrimidine, as we had done earlier to obtain 1a and other 5′-(ω-carboxyalkoxy) analogs from ω-bromoalkanoic acids, efforts to apply this method using methyl 4-(bromomethyl)benzoate were thwarted by several side reactions. To circumvent this problem we chose to reverse the synthetic sequence so that formation of the diaminopyrimidine ring would be the last step. Thus, we carried out the synthesis of analogs 8 and 9 according to Scheme 2. Condensation of 3-hydroxy-6-methoxybenzaldehyde (23) with methyl 3-(bromomethyl)-benzoate (24) in the presence of K₂CO₃ and a catalytic amount of a crown ether, which procedure is described in Rosowsky, A; Forsch, R. A.; Queener, S. F. J. Med. Chem. 2002, 45, 233-241, unexpectedly gave a 79% yield of the previously unknown acetal 25 after recrystallization from MeOH; however the latter was readily converted to the desired aldehyde 26 by treatment with dilute HCl. The structure of 25 was deduced from its elemental analysis, the absence of absorbance at 1685 cm⁻¹ in the infrared spectrum, and the presence, in the ¹H NMR spectrum, of characteristic signals at δ 3.35 and δ 5.64 for the CH(OMe)₂ group. That O-alkylation had occurred in the expected manner was also evident from the presence of a peak at δ 5.07 corresponding to the OCH₂ protons. The overall two-step yield of 26 from 24 was 69%. Interestingly, when 23 was condensed with t-butyl 4-(bromomethyl)benzoate only the expected aldehyde 28 was obtained even though the product was recrystallized from MeOH. The probable reason for this difference in behavior between the meta and para esters is that dilute citric acid was used during the workup of the reaction of 24 but not 27. Thus it is possible that some residual citric acid was present during the recrystallization from MeOH. The remaining steps were performed according to one of the standard routes TMP analogs, in which the aldehyde is treated sequentially with 3-morpholinopropionitrile, aniline hydrochloride, and guanidine (Kompis, I.; Wick, A. Helv. Chim. Acta 1977, 60, 3025-3034; and Roth, B.; Baccanari, D. P.; Sigel, C. W.; Hubbell, J. P.; Eaddy J.; Kao, J. C.; Grace, M. E.; Grace, M. E.; Rauckinan, R. S. J. Med. Chem. 1988, 31, 122-129), and in this case NaOH to cleave the ester groups to acids. Although none of the intermediates in the four-step sequence from 26 and 28 to the final products 8 and 9, respectively, were isolated in pure state before proceeding to the next step, the final acids were purified by preparative HPLC on C₁₈ silica gel using 18% MeCN in 0.1 M NH₄OAc, pH 7.4, as the eluent. Large amounts of nonpolar impurities were easily removed in this manner, and the identity and purity of the products were confirmed established by microchemical and ¹H NMR analysis. As expected, 8 and 9 were soluble in dilute aqueous base, and precipitated at a weakly acidic pH.

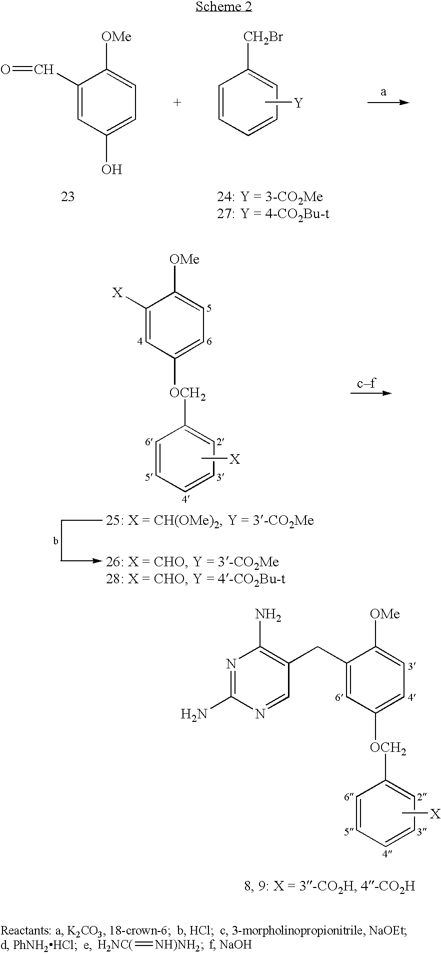

The final two compounds in this series of analogs, 10 and 11, were synthesized according to Scheme 3, starting from ethyl salicylate (29) and ethyl 3-hydroxybenzoate (30), respectively. Condensation of the phenols with propargyl bromide in the presence of K₂CO₃ and a crown ether catalyst in DMF afforded the O-propargyl ethers 31 and 32. Further reaction of the ethers with iodide 19 in the presence of (Ph₃P)₂PdCl₂, CuI, and Et₃N, followed by saponification, afforded the desired acids in overall two-step yields of 21% and 24%. Neither 31 and 32 nor the ester intermediates 33 and 34 from the Sonogashira reaction were chromatographed, but the final products were carefully purified by preparative HPLC (C₁₈ silica gel, 20% MeCN in 0.1 MNH₄OAc, pH 7.4) and characterized by their ¹H NMR spectra in DMSO-d₆ solution, which displayed the expected resonance signals at δ 3.50 (benzylic CH₂), 3.82 (OMe), 5.04 (CH₂O), and 7.40 (pyrimidine 6-H) in addition to appropriate peaks for the various protons of the phenyl rings. 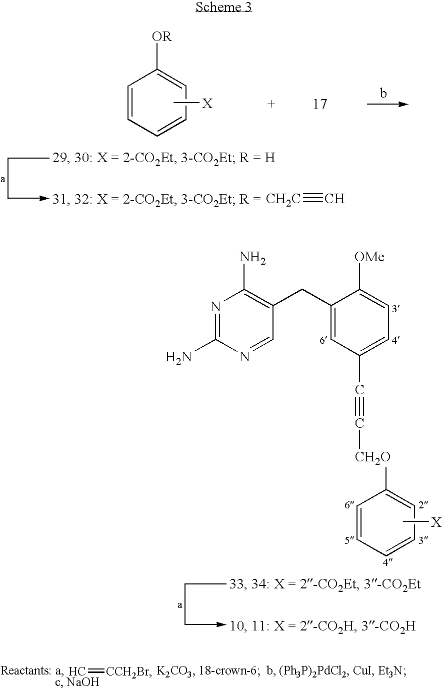 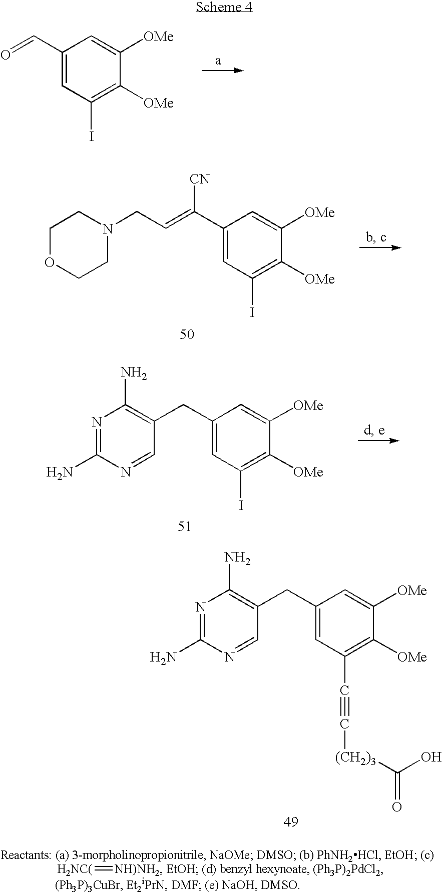
Enzyme Inhibition Assays

Compounds 2-11 were tested for the ability to inhibit Pc, Tg, and Ma DHFR according to the standardized spectrophotometric assays used in the Queener laboratory, and their potencies and selectivities were compared with updated values for 1a. 1b, TMP, and PTX. See, for example, Broughton, M. C.; Queener, S. F. Antimicrob. Agents Chemother. 1991, 35, 1348-1355; and Chio, L.-C.; Queener, S. F. Antimicrob. Agents Chemother. 1993, 37, 1914-1923. The results are presented in Table 1, and discussed for each of enzyme in the following sections.

### Rest of this Page Intentionally Left Blank

TABLE 1
inhibition of P. carinii (Pc), T. gondii (Tg), M. avium (Ma), and rat dihydrofolate reductase by
2,4-diamino-5-(2′-methoxy-5′-substituted benzyl)pyrimidines.

| IC₅₀ (μM)^{a} | | | | | Selectivity Index (SI)^{b} | | |
|---|---|---|---|---|---|---|---|
| Cmpd | P. carinii | T. gondii | M. avium | Rat | P. carinii | T. gondii | M. avium |
| 1a | 0.25 | 0.18 | 0.0048 | 2.7 | 11 (8.5-13) | 16 (12-19) | 560 (430-730) |
| | (0.21-0.30) | (0.15-0.20) | (0.0040-0.0059) | (2.502.9) | | | |
| 1b | 0.054^{c} | 0.11 | 0.058^{c,d} | 4.6^{e} | 85 (79-97) | 42 (19-72) | 79 (19-230) |
| | (0.045-0.068) | (0.066-0.17) | (0.02-0.16) | (3.2-6.0) | | | |
| 2 | 0.13 | 0.097 | 0.0044 | 4.0 | 31 (24-39) | 41 (34-39) | 900 (700-1100) |
| | (0.12-0.15) | (0.093-0.10) | (0.0040-0.0049) | (3.5-4.6) | | | |
| 3 | 0.028 | 0.032 | 0.007 | 2.2 | 79 (63-100) | 69 (52-91) | 280 (200-380) |
| | (0.025-0.03) | (0.027-0.037) | (0.0064-0.0095) | (1.9-2.5) | | | |
| 4 | 0.87 | 0.072 | 0.041 | 25 | 28 (19-42) | 340 (230-510) | 590 (330-1100) |
| | (0.77-0.98) | (0.064-0.081) | (0.31-0.56) | (19-32) | | | |
| 5 | 0.15 | 0.058 | 0.035 | 3.2 | 21 (14-31) | 54 (39-77) | 91 (51-160) |
| | (0.13-0.18) | (0.052-0.065) | (0.025-0.048) | (2.5-4.0) | | | |
| 6 | 0.15 | 0.0084 | 0.016 | 4.1 | 27 (18-41) | 490 (340-700) | 260 (140-460) |
| | (0.13-0.17) | (0.075-0.009) | (0.011-0.022) | (3.1-5.3) | | | |
| 7 | 0.53 | 0.030 | 0.089 | 4.6 | 8.6 (6.4-11) | 150 (120-190) | 52 (38-70) |
| | (0.43-0.65) | (0.026-0.034) | (0.071-0.11) | (4.2-5.0) | | | |
| 8 | 0.89 | 0.60 | 0.12 | 19 | 21 (13-33) | 31 (21-46) | 150 (100-230) |
| | (0.73-1.1) | (0.53-0.69) | (0.1-0.14) | (14-24) | | | |
| 9 | 1.2 | 2.0 | 0.060 | 21 | 17 (9.6-30) | 11 (6.4-18) | 340 (210-560) |
| | (0.098-1.5) | (1.7-2.2) | (0.053-0.069) | (13-29) | | | |
| 10 | 2.3 | 0.50 | 0.036 | 6.2 | 2.7 (2.1-3.5) | 13 (9.3-17) | 170 (130-230) |
| | (2.1-2.5) | (0.44-0.56) | (0.032-0.041) | (5.2-7.4) | | | |
| 11 | 5.6^{e} | 1.6 | 0.057 | 14 | 2.4 (1.6-3.7) | 8.3 (4.6-15) | 240 (170-250) |
| | (4.4-7.2) | (1.1-2.4) | (0.050-0.065) | (11-16) | | | |
| 49 | 0.0010 | 0.034 | 0.0024 | 5000 | 5000 (3600-7100) | 150 (110-190) | 2100 (2000-2800) |
| | (0.00082-0.0012) | (0.030-0.039) | (0.0021-0.0027) | (4300-5800) | | | |
| TMP^{f} | 13 | 2.8 | 0.30 | 180 | 14 (10-20) | 65 (48-87) | 610 (460-810) |
| | (10-16) | (2.4-3.3) | (0.26-0.35) | (160-210) | | | |
| PTX^{f} | 0.013 | 0.0043 | 0.00061 | 0.0033 | 0.26 (0.17-0.42) | 0.76 (0.63-0.97) | 5.4 (4.1-7.2) |
| | (0.009-0.017) | (0.0040-0.0046) | (0.0053-0.0007) | (0.0029-0.0039) | | | |

^{a}Numbers in parentheses are 95% confidence limits. The difference in IC₅₀ between rat liver and each of the parasite enzymes was determined to be statistically significant at P < 0.01.
^{b}SI = IC₅₀ (Pc, Tg, or Ma). Numbers in parenthesis are 95% confidence limits, and represent a range calculated by dividing the lower end of the 95% confidence interval for rat liver DHFR by the high end of the 95% confidence interval for Pc, Tg, or Ma DHFR. SI values exceeding 100 are rounded off to two figures. Non-selectivity is denoted by an SI of <1.0.
^{c}Mean of three experiments on different days.
^{d}The IC₅₀ of 1b against Ma DHFR was inadvertently recorded as 0.0058 μM in Rosowsky, A; Forsch, R. A.; Queener, S. F. J. Med. Chem. 2002, 45, 233-241; as result, the SI calculated therein for this compound was off by a factor of 10.
^{e}Mean of two experiments on different days.
^{f}TMP = trimethoprim; data taken from Rosowsky, A; Forsch, R. A.; Queener, S. F. J. Med. Chem. 2002, 45, 233-241.
^{g}PTX = piritrexim. Historical IC₅₀ for PTX, obtained with an older sample and cited previously for 1 were obtained for comparison purposes were the following: Pc DHFR, 0.031 μM; Tg DHFR, 0.017 μM; rat liver, 0.0015 μM.
The data in Table 1 were obtained with a newer sample of PTX and the IC₅₀ of PTX against Ma DHFR had not been determined previously.

Pneumocystis carinji DHFR

As shown in Table I, the most potent inhibitor of Pc DHFR among this group was the 5′-(5-carboxy-1-pentynyl) analog 3, with an IC₅₀ of 0.028 μM. Thus, against the enzyme from this organism, 3 was slightly more potent than 1b. However, because potency increased by about the same extent against rat DHFR, the selectivity index (SI) as defined in Table 1, footnote b, was not changed significantly. Although there was no substantial improvement in potency or selectivity, this finding was nonetheless of interest because it demonstrated that the oxygen atom at the 5′-position of 1b can be safely replaced by a carbon-carbon triple bond where Pc DHFR binding is concerned. It may be noted that the C≡C(CH₂)₃CO₂H side chain in 3 contains the same number of atoms as the O(CH₂)₄CO₂H side chain in lb, but is less flexible because of the geometric constraints imposed by the triple bond. From the enzyme binding data, it appears that the terminal carboxyl group may nonetheless be adequately positioned in the active site to interact with Arg75 (Pc numbering). Of interest in this regard is that the shorter analog 2 was a slightly weaker inhibitor of both Pc and rat DHFR than 3, a pattern also observed in the previous series of 5′-(ω-carboxyalkoxy) analogs. See, Rosowsky, A; Forsch, R. A.; Queener, S. F. J. Med. Chem. 2002, 45, 233-241. However compound 4 displayed a more striking decrease in potency than either 2 or 3 against both the Pc and rat enzymes. Finally, with regard to the potency and selectivity of the alkynes 5-7 versus the alkanes 2-4, a dramatic pattern of structure-activity relationships did not emerge, and we were not able to determine whether a carboxyalkyne or carboxyalkane side chain was superior. With respect to compounds 8-11 as inhibitors of Pc versus rat DHFR, it can be seen from Table 1 that they are all considerably weaker than 1b or 3 against the Pc enzyme, and that the potency difference is greater against this enzyme than against the rat enzyme, resulting in an overall decrease in selectivity. At least with these examples, therefore, it appears that the introduction of a bulky second phenyl ring in the side chain, together with replacement of the aliphatic COOH group by an aromatic one, was unfavorable. It may be noted that, when the total number of side-chain atoms is added up, the number of atoms separating the carboxyl group from the 2′-methoxyphenyl ring is five in 8, six in 9 and 10, and seven in 11. The corresponding number of atoms is four in 2 and 5, five in 3 and 6, and six in 4 and 7. Thus, among the analogs with an extra phenyl ring, 8-10 most closely approximate 1b and 2-7 in terms of the total number of atoms (aromatic and aliphatic) separating the carboxyl group from the rest of the molecule, whereas in 11 there is one additional atom.

Toxoplasma gondii DHFR

In contrast to Pc DHFR, the most potent inhibitor of the Tg enzyme was 6, with an IC₅₀ of 0.0084 μM (Table 1). Whereas this corresponded to a roughly one-log increase in binding relative to 1b, a comparable effect was not observed with rat DHFR. As a result, the selectivity index (SI) of 6 was 490 as compared with a value of only 42 in the case of 1b. Thus, replacement of the oxygen atom at the 5′-position of 1b by a saturated carbon resulted in a considerable improvement in both potency and selectivity. Interestingly, this was in contrast to the decreased potency and selectivity of 6 relative to 1b against Pc DHFR and of 6 relative to 1a against Ma DHFR.

The published literature on Tg DHFR inhibitors contains a large number of mono-, di-, and tricyclic 2,4-diaminopyrimidines that are much more potent than TMP, but only a handful that are both more potent and more selective against Tg DHFR versus rat DHFR under our standardized assay conditions. As summarized in Table 2, these compounds include 35 (Rosowsky, A; Forsch, R. A.; Queener, S. F. J. Med. Chem. 2002, 45, 233-241), a two-carbon homolog of 1, the TMP analog 36 (epiroprim) (Gangjee, A.; Vasudevan, A.; Queener, S. F.; Kisliuk, R. L. J. Med. Chem. 1996, 39, 1438-1446), the 2,4-diamino-5-aryl-6-ethylpyrimidines 37 and 38 (Stevens, M. F. G.; Phillip, K. S.; Rathbone, D. L.; O'Shea, D. M.; Queener, S. F.; Schwalbe, C. H.; Lambert, P. A. J Med. Chem. 1997, 40, 1886-1893), the-2,4-diamino-6-substituted pteridines 39 (Piper, J. R.; Johnson, C. A.; Krauth, C. A.; Carter, R. L.; Hosmer, C. A.; Queener, S. F.; Borotz, S. E.; Pfefferkorn, E. R. J Med Chem. 1996, 39, 1271-1280.) and 40 (Rosowsky, A.; Cody, G.; Galitsky, N.; Fu, H.; Papoulis, A. T.; Queener, S. F. J. Med. Chem. 1999, 42, 4853-4860.), the 2,4-diamino-6-benzylaminopyrido[2,3-d]pyrimidine 41 (albeit only when tested against Tg versus human instead of rat DHF, and under assay conditions different from those typically used with the other compounds reported; see, Gangjee, A.; Vasudevan, A.; Queener, S. F.; Kisliuk, R. L. J. Med. Chem. 1996, 39, 1438-1446), and the 2,4-diamino-5-substituted pyrrolo[2,3-d]pyrimidines 42 and 43(Gangjee, A.; Mavandadi, F.; Queener, S. F.; McGuire, J. J. J. Med. Chem. 1995, 38, 2158-2165). Very surprisingly, three 2,4-diamino-6,7-disubstituted pteridines (44-46) have also been reported to be among this select group (Chio, L.-C.; Queener, S. F. Antimicrob. Agents Chemother. 1993, 37, 1914-1923). Bulky substitution at the 7-position of 2,4-diaminopteridines is generally considered very unfavorable for DHFR binding. However it is possible that, in the case of the Tg enzyme, the selectivity of these compounds relative to rat DHFR reflects the fact that the Tg enzyme is a larger difunctional protein containing both a thymidylate synthase (TS) and DHFR domain (Roos, D. S. J. Biol. Chem. 1993, 268, 6269-6280). This question will presumably remain unanswered until the 3D structure of the Tg enzyme is fully solved. On the basis of its 330-fold increase in potency and 7.5-fold increase in selectivity relative to TMP against Tg DHFR, and in light of its very good profile when compared to the inhibitors shown in Table 2, compound 6 may be viewed as a most interesting lead for further structure-activity optimization.

TABLE 2
Various compounds of the invention and the literature possessing inhibitory activity against Tg DHFR.

| | |
|---|---|
| 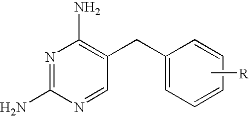 | 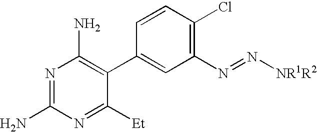 |
| 6: R = 2′-OMe-5′-[(CH₂)₅CO₂H (IC₅₀ = 0.0084 μM, SI = 490) | 37: R¹R² = morpholino (IC₅₀ = 0.19 μM, SI = 140)¹⁸ |
| 35: R = 2′-OMe-5′-O(CH₂)₆CO₂H (IC₅₀ =0.18 μM, SI = 65)² | 38: R¹ = R² = Me (IC₅₀ = 0.31 μM, SI = 61)¹⁸ |
| 36: R = 3′,5′-(OMe)₂, 4′-(1-pyrrolyl)(IC₅₀ = 0.47 μM, SI = 160)¹⁷ | |
| 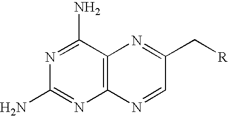 | 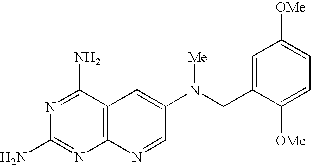 |
| 39: R = CH₂SC₆H₅ (IC₅₀ = 0.77 μM, SI = 319)¹⁹ | 41 (IC₅₀ = 0.028 μM, SI = 304)¹⁷ |
| 40: R = N-Dibenz[b,f]azepinyl (IC₅₀ = 0.043 μM, SI = 102)^{3g} | |
| 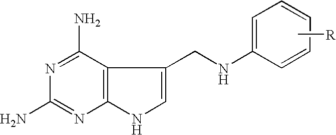 | 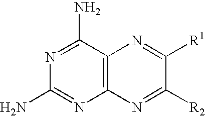 |
| 42: R = 2′,5′-(OMe)₂ (IC₅₀ = 1.7 μM, SI = 92)²⁰ | 44: R¹ = R² = C₆H₅ (IC₅₀ = 0.24 μM, SI = 58)¹⁶ |
| 43: R =2′,3′-(CH₂)₄ (IC₅₀ = 1.1 μM, SI = 54)²⁰ | 45: R¹ = 3-MeC₆H₄, R² = NH₂ (IC₅₀ = 0.018 μM, SI = 47)¹⁶ |
| | 46: R¹ = COC₆H₅, R² = C₆H₅ (IC₅₀ = 0.86 μM, SI = 66)¹⁶ |

Mycobacterium avium DHFR

As indicated in Table 1, compound 2 was a much better inhibitor of Ma DHFR than TMP, and was similar to the previously reported 5′-O-(5-carboxybutyloxy) analog 1a. Indeed, with its nearly 3-log selectivity for Ma DHFR relative to the rat enzyme and its approximately 70-fold superiority over TMP in terms of potency, 2 ranks among the best inhibitors of Ma DHFR described to date. Although it stands out among the other compounds tested, there were also several analogues with respectable SI values in the 200-600 range. However, it should be noted that high SI value were not always accompanied by high potency. Thus, while the calculated SI of 590 listed for compound 4 had a range (330-1100) that overlapped that of 2 (700-1100), its potency against both Ma and rat DHFR was considerably lower.

Suling and coworkers recently published data on a library of almost eighty 2,4-diaminopyrido[2,3-d]pyrimidines with either arylamino or arylthiomethyl groups at the 6-position as inhibitors of Ma versus human DHFR. See, Suling, W. J.; Seitz, L. E.; Pathak, V.; Westbrook, L.; Barrow, E. W.; Zywno-Van Ginkel, S.; Reynolds, R. C.; Piper, J. R.; Barrow, W. W. Antimicrob. Agents Chemother. 2000, 44, 2784-2793. The most potent member of their series against Ma DHFR was 2,4-diamino-6-(2′-methyl-4′-chloroanilino)methyl-5-methylpyrido[2,3-d]pyrimidine (47), with an IC₅₀ of 0.00019 μM. However, because 47 was also a potent inhibitor of a mammalian DHFR (human in this case), its SI value was only 15. A second compound, 2,4-diamino-6-(2′,5′-diethoxy-anilino)methyl-5-methylpyrido[2,3-d]pyrimidine (48), on the other hand, had IC₅₀ values of 0.00084 and 2.3 μM against Ma and human DHFR, respectively. Unfortunately an IC₅₀ value for 48 against rat DHFR was not reported, and the assay conditions differed in some respects from those used routinely by the Queener laboratory. The binding of inhibitors to DHFR can vary substantially depending on the pH, the temperature, and other variables. Thus, it remains to be determined how 2 and 48 would compare if they were tested under identical assay conditions in the same laboratory. Irrespective of the outcome of such a comparison, the excellent combination of potency reported here over a range of 2,4-diamino-5-(substituted benzyl)pyrimidines, including some that are not particularly potent or selective against Pc or Tg DHFR, demonstrates that potency and selectivity against Ma DHFR does not require the inhibitor to be a ftused-ring 2,4-diamino-pyrimidine.

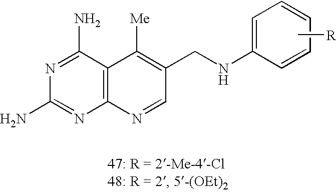

The 3D structure of the ternary complex of Ma DHFR with NADPH and TMP has not been reported. However it is noteworthy that, in a recent study of the closely related enzyme from M. tuberculosis by X-ray crystallography, Li and coworkers suggested that TMP analogs with extended hydrophobic substitution on the benzyl ring might be designed to selectively fit into a binding pocket that appears to not be available in the active site of mammalian DHFR enzymes. See, Li, R; Sirawaporn, R.; Chitnumsub, P.; Sirawaporn, W.; Wooden, J.; Athappilly, F.; Turley, S.; Hol, W. G. J. Mol. Biol 1999, 295, 307-323. Although they are 2′,5- rather than 3′,4′,5-trisubstituted benzyl derivatives, it would be of interest to determine whether 2-4 can bind to the Ma enzyme in such a way as to allow the hydrocarbon portion of the 5′-(ω-carboxy-1-alkynyl) side chain to make van der Waals contact with this hydrophobic domain.

A variety of small-molecule antifolates of the 2,4-diamino-5-(substituted benzyl)-pyrimidine family, each more potent and/or more selective than TMP against at least one of three different opportunistic pathogens of AIDS, have been discovered in the development of the present invention. For example, 2,4-diamino-5-[5′-(4-carboxy-1-pentynyl)-2′-methoxybenzyl]pyrimidine (3) inhibited Pc DHFR with a selectivity index of 79 relative to the rat liver enzyme, and was 430 times more potent than TMP. Another compound of the invention, 5-[5′-(5-carboxy-1-butynyl)-2′-methoxybenzyl]pyrimidine (2), with one less carbon than 3 in the side chain, had a selectivity index of 910 against Ma DHFR and was 43 times more potent than TMP. The third compound, 2,4-diamino-5-[5′-(5-carboxypentyl)-2′-methoxy-benzyl]pyrimidine (6) had a selectivity index of 490 against Tg DHFR, and was 320 times more potent than TMP. A fourth compound of the invention, 2,4-diamino-5-[5′-(6-carboxy-1-hexynyl)-2′-methoxy-benzyl]pyrimidine (4), provides a selectivity index of >300 against both Tg and Ma DHFR notwithstanding the lower potency of compound 4 against all three of the parasite enzymes (Pc, Tg, and Ma) when compared to the level of inhibition for compounds 3 and 6.

The following non-limiting examples are illustrative of the invention. All documents mentioned herein are incorporated herein by reference in their entirety.

General Experimental Comments

IR spectra were obtained on a Perkin-Elmer Model 781 double-beam recording spectrophoto-meter. Only peaks with wave numbers above 1200 cm⁻¹ are reported. ¹H NMR spectra were recorded in DMSO-d₆ solution at 200 MHz on a Varian VX200 instrument. Each peak is denoted as a singlet (s), broad singlet (br s), doublet (d), doublet of doublets (dd), triplet (t), doublet of triplets (dt), or pentet (p). Integrated peak areas are not listed when the resonance signal was partly obscured by water or DMSO, or in the case of NH₂ groups on the pyrimidine ring. Signals for the aromatic protons in compounds with two phenyl rings are identified according to the numbering in Schemes 2 and 3. TLC analyses were on Whatman MK6F silica gel plates with UV illumination at 254 nm. Column chromatography was on Baker 7024 flash silica gel (40 μm particle size). HPLC separations were performed on C₁₈ radial compression cartridges (Millipore, Milford, Mass.; analytical, 5 μm particle size, 5×100 mm; preparative, 15 μm particle size, 25×100 mm). Melting points were measured in Pyrex capillary tubes in a Mel-Temp apparatus (Fisher, Pittsburgh, Pa.), and are not corrected. 3-Morpholinopropionitrile was prepared by adding acrylonitrile dropwise to an equimolar amount of morpholine in an ice-bath, and stirring the mixture at room temperature for 1 h. The resulting light-yellow oil did not require purification and was used directly. 2,4-Diamino-5-(2′-methoxybenzyl)pyrimidine (18) and 2,4-diamino-5-(5′-iodo-2′-methoxybenzyl)pyrimidine (19) were prepared according to the literature: 18, 42% yield, mp 160-161° C. (lit. 160 ° C.); 19, 66% yield, mp 207-208° C., lit. 205° C.). Other chemicals were purchased from Sigma-Aldrich (St. Louis, Mo.), Acros Organics (Pittsburgh, Pa.), or Lancaster Synthesis (Windham, N.H.). Elemental analyses were performed by Quantitative Technologies, Inc. (Whitehouse, N.J.), and were within ±0.4% of theoretical values. Where microanalytical results were consistent with residual acetic acid, its presence in the sample was confirmed by the finding of a methyl signal at δ 1.9 in the ¹H NMR spectrum.

### EXAMPLES

### Example 1

### 2,4-Diamino-5-[5′-(4-carboxy-1-butynyl)-2′-methoxy)benzyl]pyrimidine (2)

Step 1. Benzyl 4-pentynoate (15) was prepared by stirring a solution of 4-pentynoic acid (12) (1.96 g, 0.02 mol) in dry DMF (25 mL) with Cs₂CO₃ (3.25 g, 0.01 mol) for 10 min, followed by addition of benzyl bromide (2.38 mL, 3.42 g, 0.02 mol). After 18 h of stirring at room temperature, the solvent was removed by rotary evaporation (vacuum pump) and the residue was partitioned between EtOAc and H₂O. Evaporation of the organic layer gave 15 as an oil whose NMR spectrum showed that it still contained some DMF and a trace of benzyl bromide but was suitable for use in the next step.

Step 2. A stirred mixture of 15 (1.3 g, estimated by NMR to contain ca. 6.0 mmol), iodide 19 (1.42 g, 4.0 mmol), (Ph₃P)₂PdCl₂ (15 mg), CuI (1 mg), and Et₃N (5 mL) in dry DMF (5 mL) was heated at 60° C. for 20 h. A clear solution formed after ca. 1 h. The solvent was removed by rotary evaporation, and the residue was triturated successively with isooctane and H₂O. Recrystallization from EtOH afforded 20 as a light-yellow powder (1.59 g, mp 208° C.) which was used in the next step without additional purification.

Step 3. A solution of 20 (416 mg, 1.0 mmol) in dry DMSO (4 mL) was treated dropwise with 1 N NaOH with swirling. The mixture was diluted immediately with H₂O (40 mL), acidified with 10% AcOH, and chilled in ice. The precipitate was collected and dried to obtain a beige solid; crude yield 239 mg. Analytically pure 2 for bioassay was obtained by preparative HPLC (C₁₈ silica gel, 13% MeCN in 0.1 M NH₄OAc, pH 7.4). Appropriately pooled fractions were concentrated and freeze-dried; yield 123 mg (32% based on the amount of 19 used); mp 162-163° C. (softening without giving a true melt); IR (KBr) ν 3500-2800 (broad), 3350, 3210, 2930, 1665, 1560-1530, 1505, 1460, 1405, 1295, 1115 cm⁻¹; ¹H NMR (DMSO-d₆) δ 2.50 (m, 4H, CH₂CH₂, partly overlapped by DMSO peak), 3.49 (s, 2H, bridge CH₂), 3.87 (s, 3H, OMe), 5.84 (br s, NH₂), 6.16 (br s, NH₂), 7.00 (m, 2H, aryl 3′- and 6′-H), 7.22 (d, J=9 Hz, 1H, aryl 4′-H), 7.39 (s, 1H, pyrimidine 6-H). Anal. (C₁₈H₂₀N₄O₃.0.5CH₃CO₂H.0.2H₂O) Calc. C, 61.02; H, 6.04; N, 14.98; Found C, 60.93; H, 5.93; N, 14.98.

### Example 2

### 2,4-Diamino-5-[5′-(4-carboxy-1-pentynyl)-2′-methoxy)benzyl]pyrimidine (3)

Step 1. Benzyl 5-hexynoate (16) was prepared by stirring a solution of 5-hexynoic acid (13) (1.12 g, 0.01 mol) in dry DMF (25 mL) with Cs₂CO₃ (1.63 g, 0.005 mol) for 10 min, followed by addition of benzyl bromide (1.19 mL, 1.71 g, 0.01 mol). After 18 h of stirring at room temperature, the solvent was removed by rotary evaporation (vacuum pump) and the residue was partitioned between EtOAc and H₂O. Evaporation of the organic layer gave 16 as an oil suitable for use directly in the next step.

Step 2. A stirred mixture of 16 (0.65 g, estimated by NMR to contain ca. 36.0 mmol), iodide 19 (1.42 g, 4.0 mmol), (Ph₃P)₂PdCl₂ (10 mg), CuI (1 mg), and Et₃N (3 mL) in dry DMF (3 mL) was heated at 60° C. for 72 h. The solvent was removed by rotary evaporation, and the residue was triturated successively with isooctane and H₂O. Attempted recrystallization from EtOH gave only a gum. Therefore the solvent was evaporated, and the residue was re-dissolved in hot THF (40 mL). A small amount of insoluble material was filtered off, the filtrate was concentrated to dryness, and the residue, containing ester 21, was taken up directly in dry DMSO (6 mL). The solution was treated dropwise with 1 N NaOH (6 mL), then diluted with H₂O (100 mL) and chilled. A small amount of residual non-saponifiable material was removed by filtration, and the filtrate was acidified with 10% AcOH. The precipitate was collected and dried in a lyophilization apparatus to obtain a beige solid. Attempted recrystallization from hot MeOH led to separation of a brown insoluble gum suggesting that decomposition was occurring. The methanolic solution was immediately decanted and left to cool passively in the hood at room temperature, producing a first crop of analytically pure 3 as white crystals weighing 32 mg (5%); mp 121-124° C. (softening without giving a true melt); IR (KBr) ν 3500-2800 (broad), 3350, 3210, 2930, 1665, 1560-1530, 1505, 1460, 1405, 1295, 1115 cm⁻¹; ¹H NMR (DMSO-d₆) δ 1.72 (p, J=7 Hz, 2H, C≡CCH₂CH₂CH₂), 2.37 (two overlapping t, 4H, C≡CCH₂CH₂ CH₂), 3.50 (s, 2H, bridge CH₂), 3.80 (s, 3H, OMe partly overlapped by H₂O), 5.86 (br s, NH₂), 6.20 (br s, NH₂), 6.95 (m, 2H, aryl 3′- and 6′-H), 7.24 (dd, J=8 Hz, J=2 Hz, 1H, aryl 4′-H), 7.39 (s, 1H, pyrimidine 6-H). Another 153 mg (24%) of less pure 3 was obtained from the mother liquor and used for hydrogenation as described below. Anal. (C₁₈H₂₀N₄O₃.0.5AcOH.0.2 H₂O) Calc. C, 54.51; H, 6.26; N, 14.61; Found C, 54.67; H, 6.01; N, 14.65.

### Example 3

### 2,4-Diamino-5-[5′-(6-carboxy-1-hexynyl)-2′-methoxy)benzyl]pyrimidine (4)

Step 1. Benzyl 6-heptynoate (17) was prepared by stirring a solution of 6-heptynoic acid (14) (1.26 g, 0.01 mol) in dry DMF (15 mL) with Cs₂CO₃ (1.63 g, 0.005 mol) for 10 min, followed by addition of benzyl bromide (1.19 mL, 1.71 g, 0.01 mol). After 18 h of stirring at room temperature, the solvent was removed by rotary evaporation (vacuum pump) and the residue was partitioned between EtOAc and H₂O. Evaporation of the organic layer gave 17 as an oil suitable for use directly in the next step.

Step 2. A stirred mixture of 17 (0.65 g, estimated by NMR to contain ca. 3.0 mmol), iodide 19 (0.71 g, 2.0 mmol), (Ph₃P)₂PdCl₂ (20 mg), CuI (2 mg), and Et₃N (5 mL) in dry DMF (5 mL) was heated at 65° C. for 2.5 h. Additional amounts of (Ph₃P)₂PdCl₂ (10 mg), CuI (1 mg) were added, and heating was continued for 18 h. The solvent was evaporated under reduced pressure, and the residue was triturated successively with isooctane and H₂O. The solid was taken up in DMSO (5 mL) and treated with a solution of NaOH (220 mg, 4.4 mmol) in H₂O (1 mL). The solution was diluted to 80 mL with H₂O, then chilled, adjusted to ca. pH 8 with 10% AcOH, and filtered to remove a trace of insoluble material. The filtrate was subjected to preparative HPLC (C₁₈ silica gel, 20% MeCN in 0.1 MNH₀OAc, pH 7.4, 10 mL/min) and fractions eluting in the major peak (ca. 12 min on the analytical column at 1.0 mL/min) were pooled and freeze-dried to obtain 4 as a white solid; 292 mg (36%); mp 187-189° C. (softening without giving a true melt); IR (KBr) ν 3370, 3220, 2950, 2880, 2850br, 1690sh, 1670, 1565, 1540, 1505, 1465, 1450sh, 1430, 1405, 1375s, 1340, 1320, 1295, 1350, 1300 cm⁻¹; ¹H NMR (DMSO-d₆) δ 1.63 (m, 4H, CH₂CH₂CH₂CO₂H), 2.18 (t, J=7 Hz, 2H, CH₂CO₂H), 2.39 (t, J=7 Hz, C≡CCH₂, 3.80 (s, OMe, partly overlapped by broad H₂O peak), 5.74 (br s, NH2), 6.09 (br s, NH₂), 6.93 (d, J=8 Hz, 1H, 3′-H), 7.02 (d, J=2 Hz, 1H, 6′-H), 7.22 (dd, J=8 Hz, J=2 Hz, 1H, 4′-H), 7.41 (s, 1H, pyrimidine 6-H). The signal for the CH₂ bridge was completely obscured by a strong H₂O peak. Anal. (C₁₉H₂₂N₄O₃.2.75H₂O) Calc. C, 56.49; H, 6.86; N, 13.87; Found C, 56.71; H, 6.61; N, 13.66.

### Example 4

### 2,4-Diamino-5-[5′-(4-carboxybutyl)-2′-methoxy)benzyl]pyrimidine (5)

A solution of recrystallized 20 (416 mg, 1.0 mmol) in dry DMF (20 mL) containing 10% Pd—C (100 mg) was shaken under H₂ (50 psi initial pressure) for 18 h. After filtration to remove the catalyst, the solution was evaporated to a solid which was not soluble in NaOH, indicating that the ester group had survived. The solid, assumed to be the benzyl ester of 5, was dissolved directly in hot EtOH (20 mL) and treated with 1 N NaOH (2 mL). The solvent was evaporated under reduced pressure, and the residue was taken up in H₂O. A trace of cloudiness, indicating that a trace of ester was still present, was discharged by adding another small portion of 1 N NaOH and some EtOH. The volume was reduced by rotary evaporation, and 10% AcOH was added dropwise until a solid formed, which was filtered, and dried on a lyophilizer to obtain 5 as a white powder (117 mg, 35%); mp 134-137° C. (softening without giving a true melt); IR (KBr) ν 3330, 3180, 2930, 2850, 1660, 1560, 1505, 1455, 1400, 1290, 1250 cm⁻¹; ¹H NMR (DMSO-d₆) δ 1.46 (m, 4H, CH₂CH₂CH₂CO₂H), 1.77 (m, 4H, CH₂CH₂CH₂CH₂CO₂H), 3.7 (s, 2H, bridge CH₂), 3.75 (s, OMe, partly overlapped by broad H₂O peak), 5.66 (br s, NH₂), 6.03 (br s, NH₂), 6.89 (m, 3H, 3′-, 4′-, and 6′-H), 7.34 (s, 1H, pyrimidine 6-H). Anal (C₁₇H₂₂N₄O₃.0.5 AcOH) Calc. C, 59.99; H, 6.71; N, 15.55; Found C, 60.14; H, 6.61; N, 15.35.

### Example 5

### 2,4-Diamino-5-[5′-(5-carboxypentyl)-2′-methoxy)benzyl]pyrimidine (6)

Benzyl 5-hexynoate (16) (0.65 g of non-purified ester, ca. 3.0 mmol, prepared from 11 as described above) was heated with iodide 19 (0.71 g, 2.0 mmol), (Ph₃P)₂PdCl₂ (10 mg), CuI (1 mg), and Et₃N (3 mL) in dry DMF (3 mL) under N₂ at 60° C. for 3.5 h. Solution occurred after ca. 1 h. The solvent was evaporated under reduced pressure, the residue was taken up in warm 95% EtOH (60 mL), and the solution, containing ester 21, was treated with 1 M NaOH (6 mL). The EtOH was. evaporated, and replaced with H₂O (50 mL). The mixture was chilled and filtered, and the filtrate was transferred directly to a Parr apparatus and subjected to catalytic hydrogenation (42 psi initial pressure) in presence of 10% Pd—C (85 mg). The catalyst was filtered off, and the filtrate was acidified with 10% AcOH. The precipitated solid was collected and dried on a lyophilizer; yield ca. 0.4 g. Analytical HPLC (C₁₈ silica gel, 20% MeCN in 0.1 M NH₄OAc, pH 7.4, 1 mL/min) showed a major peak eluting at 12 min, along with several unidentified impurities. Preparative HPLC using the same eluent system, pooling of appropriate fractions, and freeze-drying, afforded 6 as a white powder (334 mg, 46%); mp 96-98° C. (softening without giving a true melt); IR (KBr) ν 3350, 3200, 2930, 2860, 1660, 1560, 1505, 1460, 1405, 1290, 1250 cm⁻¹; ¹H NMR (DMSO-d₆) δ 1.26 (m, 2H, CH₂CH₂CH₂CO₂H), 1.45 (p, J=7 Hz, 4H, CH₂CH₂CH₂CH₂CO₂H), 2.13 (t, J=7 Hz, 2H, benzylic CH₂), 2.45 (t, CH₂CO₂H, partly overlapped by DMSO peak), 3.48 (s, bridge CH₂, partly overlapped by broad H₂O peak), 3.76 (s, OMe, partly overlapped by broad H₂O peak), 5.66 (br s, NH₂), 6.04 (br s, NH₂), 6.90 (m, 2H, aryl 3′- and 6′-H), 7.00 (dd, J=8 Hz, J=2 Hz, 1H, aryl 4′-H), 7.35 (s, 1H, pyrimidine 6-H). Anal. (C₁₈H₂₄N₄O₂.1.25H₂O) Calc. C, 58.92; H, 7.28; N, 15.27; Found C, 59.07; H, 7.17; N, 15.40.

### Example 6

### 2,4-Diamino-5-[5′-(6-carboxyhexyl)-2′-methoxy)benzyl]pyrimidine (7)

A solution of 4 (120 mg, 0.3 mmol) in DMF (10 mL) was shaken under H₂ (initial pressure 3 atm) in the presence of 5% Pd—C in a Parr apparatus for 20 h. The catalyst was removed, and the solvent evaporated under reduced pressure. The residue, consisting of the benzyl ester of 7, was treated with a small volume of dilute NaOH, and a trace of insoluble material was filtered off. The filtrate was acidified with 10% AcOH and chilled, and the precipitate was collected and dried on a lyophilizer to obtain 7 as a white solid; 115 mg (77%); mp 101-105° C. (softening without giving a true melt); IR (KBr) ν 2860, 3210, 2950, 2870, 1670, 1565, 1510, 1470, 1410, 1295, 1260 cm⁻¹; ¹H NMR (DMSO-d₆) δ 1.23 (poorly resolved m, 4H, CH₂CH₂CH₂CH₂CO₂H), 1.45 (m, 4H, CH₂CH₂CH₂CH₂CH₂CO₂H), 2.14 (t, J=7 Hz, 2H, CH₂CO₂H), 2.43 (t, J=7 Hz, benzylic CH₂, partly overlapped by DMSO), 3.74 (s, OMe, partly overlapped by broad H₂O peak), 5.65 (br s, NH₂), 6.03 (br s, NH₂), 6.88 (m, 2H, 3′- and 6′-H), 6.98 (d, J=8 Hz, 1H, 4′-H), 7.32 (s, 1H, pyrimidine 6-H). The signal for the CH₂ bridge was obscured by a strong H₂O peak. Anal. (C₁₉H₂₆N₄O₃.AcOH.1.3H₂O) Calc. C, 57.08; H, 7.44; N, 12.68; Found C, 57.22; H, 7.16; N, 12.87.

### Example 7

### Methyl 3-(3-Formyl-4-methoxyphenozymethyl)benzoate (26)

Step 1. A stirred mixture of 3-hydroxy-6-methoxybenzaldehyde (23) (456 mg, 3.0 mmol), methyl 3-bromomethylbenzoate (24) (687 mg, 3.0-mmol), K₂CO₃ (1.04 g, 7.5 mmol), and 18-crown-6 (79 mg, 0.3 mmol) in dry DMF (10 mL) was heated at 70° C. for 20 hr. The mixture was cooled to room temperature, the salts were filtered off, and the solvent was removed by rotary evaporation using a vacuum pump. The residue was partitioned between EtOAc and dilute aqueous citric acid. Evaporation of the organic layer gave a tan solid (1.03 g), which on recrystallization from MeOH proved unexpectedly to be the dimethyl acetal 25; yield 816 mg (79%); mp 65-66° C.; IR (KBr) ν 3010, 2970, 2920, 2850, 1730, 1690w, 1615, 1595, 1505, 1470, 1455, 1440, 1405, 1380, 1370, 1290, 1235, 1210 cm⁻¹; ¹H NMR (CDCl₃) δ 3.35 (s, 6H, two OMe groups of acetal), 3.81 (s, 3H, ether OMe), 3.93 (s, 3H, ester OMe), 5.07 (s, 2H, benzylic CH₂), 5.64 (s, 1H, acetal CH), 6.82 (d, J=9H, 1H, aryl 5-H), 6.91 (dd, J=9 Hz, J=3 Hz, 1H, aryl 6-H), 7.21 (d, J=3 Hz, 1H, aryl 4-H), 7.45 (t, J=8.1 Hz, 1H, aryl 5′-H), 7.64 (d, J=7 Hz, 1H, aryl 6′-H), 7.99 (d, J=7 Hz, 1H, aryl 4′-H), 8.11 (s, 1H, aryl 2′-H). Anal. (C₁₉H₂₂O₆) Calc. C, 65.88; H, 6.40; Found C, 65.89; H, 6.11.

Step 2. The acetal 25 from the preceding step was dissolved in THF (10 mL), and the solution was cooled in an ice bath and stirred while cold 1 N HCl (10 mL) was added dropwise, After 25 min at 0° C., the mixture was diluted with isooctane. Partial precipitation occurred, but when TLC showed that both the solid and the solution contained an identical single spot (R_{f}0.5, silica gel, 1:1 EtOAc-isooctane) they were re-combined in EtOAc, and the solution washed with 5% NaHCO₃. Evaporation of the organic layer afforded a solid (0.7 g crude yield), which on recrystallization from MeOH with 3 drops of added Et₃N afforded aldehyde 26 as off-white flakes (617 mg, 69%); mp 102-103° C.; IR (KBr) ν 2960w, 2880w, 1730, 1685, 1615, 1640w, 1500, 1475, 1455, 1430, 1405, 1385, 1320, 1295, 1280, 1265, 1225, 1210 cm⁻¹; ¹H NMR (CDCl₃) δ 3.91 (s, 3H, ether OMe), 3.93 (s, 3H, ester OMe), 5.09 (s, 2H, CH₂O), 6.96 (d, J=9 Hz, 1H aryl 5-H), 7.21 (dd, J=9 Hz, J=3 Hz, 1H, aryl 6-H), 7.43 (d, J=3 Hz, 1H, aryl 4-H), 7.48 (d, J=7 Hz, 1H, aryl 6′-H), 7.63 (d, J=8 Hz, 1H, aryl 5′-H), 8.01 (d, J=8 Hz, 1H, aryl 4′-H), 8.11 (s, 1H, aryl 2′-H), 10.45 (s, 1H, CH═O). Anal. (C₁₇H₁₆O₅.0.1H₂O) Calc. C, 67.59; H, 5.40; Found C, 67.42; H, 5.22.

### Example 8

### 2,4-Diamino-5-[2′-methoxy-5′-(3″-carboxybenzyloxy)benzyl]pyrimidine (8)

Step 1. Metallic Na (23 mg, 1.0 mmol) was dissolved in absolute EtOH (30 mL), and the solvent was removed by rotary evaporation and the residue re-dissolved in dry DMSO (2 mL). 3-Morpholinopropionitrile (280 mg, 2.0 mmol) was added, the reaction mixture was placed in an oil bath pre-heated to 100° C. A solution of 26 (600 mg, 2.0 mmol) in DMSO (3 mL, with slight warming as needed) was added all at once, and heating was continued for 20 min. A second portion of NaOMe (1.0 mmol in DMSO) was added, and heating was resumed for another 20 min. The reaction mixture was cooled and partitioned between EtOAc and dilute aqueous citric acid. The EtOAc layer was evaporated and the residue was dissolved in absolute EtOH (20 mL), aniline hydrochloride (389 mg, 3.0 mmol) was added, and the mixture was heated under reflux for 30 min and set aside until the next step.

Step 2. Metallic Na (184 mg, 8.0 mmol) was dissolved in absolute EtOH (25 mL), and guanidine hydrochloride (382 mg, 4.0 mmol) was added. The resulting mixture, containing some precipitated NaCl, was combined with the ethanolic solution from the preceding step. The reaction mixture was heated under reflux for 18 h, then chilled and filtered. The filter cake was taken up in H₂O, and the pH neutralized with 10% AcOH. A trace of solid precipitate, and was collected and redissolved in a small volume of 1 N NaOH. This solution was added back to the filtrate, which was then basified with 1 N NaOH (5 mL) and concentrated to dryness by rotary evaporation. The residue was taken up in H₂O (40 mL), the solution acidified with 10% AcOH and chilled, and the precipitate was collected, dried on a lyophilizer, and purified for elemental analysis and bioassay by preparative HPLC (C₁₈ silica gel, 18% MeCN in 0.1 M NH₄OAc, pH 7.4). Appropriately pooled fractions were freeze-dried, the solid was re-dissolved in 1 N NaOH. A trace of solid was filtered off, the filtrate was re-acidified with 10% AcOH and chilled, and the precipitate was collected and dried in a lyophilizer to obtain 8 as a white solid (66 mg, 9% overall yield from 26); mp ca. 140° C. (softening without giving a true melt); IR (KBr) ν 3325-3380, 2920w, 1655, 1535br, 1495, 1380, 1275 cm⁻¹; ¹HNMR (DMSO-d₆) δ 3.50 (s, bridge benzylic CH₂, partially overlapped by broad H₂O peak), 3.74 (s, OMe, partially overlapped by broad H₂O peak), 5.06 (s, 2H, CH₂O), 6.18 (br s, NH₂), 6.37(br s, NH₂), 6.83 (m, 3H, aryl 3′-, 4′-, and 6′-H), 7.32 (s, 1H pyrimidine 6-H), 7.47(t, J=8 Hz, 1H, aryl 5″-H), 7.61 (d, J=7 Hz, aryl 4″-H), 7.87 (d, J=7 Hz, 1H, aryl 6″-H), 7.99 (s, 1H, aryl 2″-H). Anal. (C₂₀H₂₀N₄O₄.0.1 AcOH.2H₂O) Calc. C, 57.43; H, 5.82; N, 13.26; Found C, 57.79; H, 5.44; N, 12.93.

### Example 9

### tert-Butyl 4-(3-formyl-4-methoxyphenoxymethyl)benzoate (28)

To a 4:1 THF-DMF mixture (10 mL) were sequentially added tert-butyl 4-bromomethylbenzoate (27) (1.08 g, 4.0 mmol), 3-hydroxy-6-methoxybenzaldehyde (23) (496 mg, 3.25 mmol),² K₂CO₃ (1.38 g, 4.0 mmol), and 18-crown-6 (106 mg, 0.4 mmol). The mixture was heated at 70° C. for 40 min, after which the THF was blown off with a stream of air. TLC (silica gel, 1:1 EtOAc-isooctane) showed some unchanged bromide (R_{f}0.6) and phenol (R_{f}0.3), along with a small new spot at R_{f}0.5 (blue-fluorescent) corresponding to the desired product. Replacement of the THF and heating for another 66 h led to complete disappearance of the phenol (R_{f}0.3). The reaction mixture was diluted with EtOAc, and the combined organic solvents were decanted from the salts, washed with H₂O, and evaporated to dryness under reduced pressure to obtain a solid (1.3 g). Recrystallization-from MeOH afforded 28 as a light-yellow powder (0.85 g, 76%); mp 91-92° C.; IR (KBr) ν 3000, 2980, 1860, 1700, 1675, 1615, 1585, 1575, 1495, 1470, 1455, 1435, 1425, 1410, 1395, 1365, 1315, 1300, 1280, 1255, 1220, 1200 cm⁻¹; ¹H NMR (CDCl₃) δ 1.60 (s, 9H, t-Bu), 3.90 (s, 3H, OMe), 5.11 (s, 2H benzylic CH₂), 6.94 (d, J=9 Hz, 1H, 5-H), 7.21 (dd, J=9 Hz, J=3 Hz, 6-H), 7.41 (d, J=3 Hz, 1H, 4-H), 7.46 (d, J=8 Hz, 2H, 2′- and 6′-H), 8.00 (d, J=8 Hz, 2H, 3′- and 5′-H). Anal. (C₂₀H₂₂O₅) Calc. C, 70.16; H, 6.48; Found C, 69.86; H, 6.35.

### Example 10

### 2,4-Diamino-5-(2′-methoxy-5′-(4″-carboxybenzyloxy)benzyl]pyrimidine (9)

Metallic Na (12 mg, 0.5 mmol) was dissolved in absolute EtOH (3 mL), and the solvent was evaporated under reduced pressure and replaced by dry DMSO (2 mL). 3-Morpholinopropionitrile (280 mg, 2.0 mmol) was added, and the mixture was placed in a bath pre-heated to 100° C., and slowly treated with a solution of 28 (684 mg, 2.0 mmol) in dry DMSO (3 mL). After another 20 min at 100° C., he reaction mixture was cooled and partitioned between EtOAc and dilute citric acid. The EtOAc layer was concentrated to dryness by rotary evaporation, and the residue was taken up in absolute EtOH (10 mL). Aniline hydrochloride (260 mg, 2.0 mmol) was added, and the reaction mixture was refluxed for 30 min. Separately, guanidine hydrochloride (478 mg, 5.0 mmol) was added to a solution of metallic Na (161 g, 7.0 mmol) in absolute EtOH (20 mL), and the mixture was swirled for 5 min and added to the foregoing ethanolic solution containing the anilino nitrile adduct. The mixture was refluxed for 18 h, then chilled and filtered. The filter cake, which was completely soluble in H₂O, was discarded, and the filtrate was evaporated under reduced pressure. The residue was taken up in trifluoroacetic acid (5 mL), the solution was evaporated to dryness, and the residue was suspended in H₂O (30 mL) and dissolved by adding 1 N NaOH and stirring for several minutes until the pH was 10. Re-acidification with 10% AcOH produced a solid, which was collected, dried in a lyophilization apparatus (crude yield 720 mg), and purified for elemental analysis and bioassay by preparative HPLC (C₁₈ silica gel, 18% MeCN in 0.1 M NH₄OAc, pH 7.4). Appropriately pooled fractions were concentrated and freeze-dried, and the solid was redissolved in dilute NH₄OH, and filtered. The filtrate was acidified with 10% AcOH, and the precipitate collected and freeze-dried to obtain 9 as a white solid (60 mg, 8% overall yield from 28); mp 250-251° C.; IR (KBr) ν 3330, 3170, 1660, 1615sh, 1595sh, 1535, 1500, 1460, 1380, 1295, 1220 cm⁻¹; ¹H NMR (DMSO)-d₆) δ 3.49 (s, bridge CH₂ partially overlapped by broad H₂O peak), 3.74 (s, OMe, partially overlapped by broad H₂O peak), 5.08 (s, 2H benzylic CH₂), 5.90 (br s, NH₂), 6.20 (br s, NH₂), 6.83 (m, 3H, 3′-, 4′-, and 6′-H), 7.37 (s, 1H, pyrimidine 6-H), 7.50 (d, J=8 Hz, 2H, 2″- and 6″-H), 7.93 (d, J=8 Hz, 3″- and 5″-H). Anal. (C₂₀H₂₀N₄O₄0.7AcOH) Calc. C, 60.85; H, 5.44; N, 13.26; Found C, 60.96; H, 5.30; N, 12.86.

### Example 11

### 2,4-Diamino-5-[2′-methoxy-5′-[3-(2″-carboxyphenoxy)propyn-1 -yl]benzyl]pyrimidine (10)

Step 1. A mixture of ethyl salicylate (29) (1.66 g, 0.01 mol), K₂CO₃ (2.07 g, 0.015 mol), a catalytic amount of 18-crown-6 (26 mg), and propargyl bromide (1.12 mL, of 80% solution in toluene, calculated to contain 1.19 g, 0.01 mol) in dry DMF (10 mL) was stirred at room temperature for 20 h. The salts were filtered off, and the solvent was evaporated under reduced pressure in a bath maintained at 50° C. The residue was partitioned between EtOAc and H₂O, and the organic layer evaporated to obtain the propargyl ether 31 (2.1 g) as an oil suitable for use directly in the next step; ¹H NMR (CDCl₃) δ 1.38 (t, J=7 Hz, 3H, CH₂CH₃), 2.53 (t, J=2 Hz), 1H, C≡CH), 4.36 (q, J=7 Hz, 2H, CH₂CH₃), 4.79 (d, J=2 Hz, 2H, ≡CCH₂O), 7.05 (dt, J=8 Hz, J=2 Hz, 1H, aryl 4-H), 7.12 (d, J=8 Hz, 1H, aryl 3-H), 7.47 (dt, J=8 Hz, J=2 Hz, 1H, aryl 5-H), 7.80 (dd, J=8 Hz, J=2 Hz, 1H, aryl 6-H).

Step 2. A mixture of propargyl ether 31 (306 mg, 1.5 mmol), iodide 19 (356 mg, 1.0 mmol), (Ph₃P)₂PdCl₂ (10 mg), CuI (1 mg), and Et₃N (3 mL) in dry DMF (3 mL) was heated under N₂ at 65° C. for 3 h. The solvent was evaporated under reduced pressure, and the residue triturated with alternating portions of isooctane and H₂O. The residue, consisting of ester 33, was dissolved in 50% EtOH (200 mL) at 50° C., and treated with Ba(OH)₂.8H₂O (947 mg, 3.0 mmol). The mixture was stirred at room temperature for 20 h, whereupon a solution a solution of (NH₄)₂CO₃ (500 mg, 5 mmol) in H₂O (10 mL) was added. After 10 min of vigorous stirring, the solid was removed by filtration and the filtrate was concentrated to a small volume under reduced pressure. Pure product for elemental analysis and bioassay was isolated by preparative HPLC (C₁₈ silica gel, 20% MeCN in 0.1 MNH₄OAc, pH 7.4, 280 nm). The major peak (eluting at 13 min on an analytical C₁₈ column using the same elution system) was concentrated and freeze-dried, and the residue dissolved in small volume of dilute NH₄OH. A small amount of insoluble material was filtered off, and the filtrate was chilled and acidified with 10% AcOH. The precipitate was collected and freeze-dried to obtain pure 10 as a white solid (104 mg, 24%); mp 147-153° C. (softening rather than giving a true melt); IR (KBr) ν 3360, 3210, 2960br, 2230, 1665, 1605, 1595, 1560, 1505, 1355, 1385, 1295, 1275sh, 1250 cm⁻¹; ¹H NMR (DMSO-d₆) δ 3.51 (s, bridge CH₂, partly overlapped by broad H₂O peak), 3.82 (s, OMe, partly overlapped by broad H₂O peak), 5.05 (s, 2H, CH₂O), 5.94 (br s, NH₂), 6.23 (br s, NH₂), 6.95-7.55 (complex m, 7H, aryl and pyrimidine protons), 7.62 (d, J=7H, 1H 3″-H). Anal. (C₂₂H₂₀N₄O₄.1.5H₂O) Calc. C, 61.25; H, 5.37; N, 12.99; Found C, 61.23; H, 5.25; N, 12.81.

### Example 12

### 2,4-Diamino-5-[2′-methoxy-5′-[3-(3″-carboxyphenoxy)-1-propynyl]benzyl]pyrimidine (11)

Step 1. A mixture of ethyl 3-hydroxybenzoate (30)(1.66 g, 0.01 mol), K₂CO₃ (2.07 g, 0.015 mol), a catalytic amount of 18-crown-6 (26 mg), and propargyl bromide (1.12 mL 1.49 g of 80% solution in toluene, calculated to contain 0.01 mol) in dry DMF (10 mL) was stirred at room temperature for 20 h. The salts were filtered off, and the solvent was evaporated under reduced pressure in a bath maintained at 50° C. The residue was partitioned between EtOAc and H₂O, and the organic layer evaporated to obtain the propargyl ether 32 (2.3 g) as an oil suitable for use directly in the next step; ¹H NMR (CDCl₃) δ 1.39 (t, J=7 Hz, 3H, CH₃CH₂), 2.53 (d, J=1 Hz, 1H, C≡CH), 4.38 (q, J=7 Hz, 2H, CH₃CH₂), 4.74 (s, 2H, ≡CCH₂O), 7.17 (d, J=8 Hz, 1H, aryl 4-H), 7.36 (t, J=8 Hz, 1H, aryl 5-H), 7.65 (m, 2H, aryl 2- and 6-H).

Step 2. A mixture of propargyl ether 32 (306 mg, 1.5 mmol), iodide 19 (356 mg, 1.0 mmol), (Ph₃P)₂PdCl₂ (10 mg), CuI (1 mg), and Et₃N (3 mL) in dry DMF (3 mL) was heated under N₂ at 65° C. for 3.5 h. The solvent was evaporated under reduced pressure, and the residue was triturated with alternating portions of isooctane and H₂O. The solid, consisting of ester 34, was collected and dissolved in DMSO (5 mL) with slight warming as needed. This solution was then swirled and treated dropwise with a solution of NaOH (120 mg, 3.0 mmol) in H₂O (1 mL). The mixture was diluted to 90 mL with H₂O, then brought to approximately pH 8 with 10% AcOH. Pure 9 for microchemical analysis and bioassay was isolated by preparative HPLC (C₁₈ silica gel, 20% MeCN in 0.1 MNH₄OAc, pH 7.4, 280 nm). Appropriately pooled eluates were concentrated and freeze-dried, the residue was re-dissolved in 0.1 N NaOH, and the solution was filtered. Acidification with 10% AcOH, followed by freeze-drying, yielded pure 9 as a white powder (100 mg, 21%); mp 149-153° C. (softening without giving a true melt); IR (KBr) ν 3360, 3200, 2960 (broad), 1670, 1610, 1560, 1505, 1460, 1445, 1390, 1275, 1245 cm⁻¹; ¹HNMR (DMSO-d₆) δ 3.51 (s, benzylic CH₂, partly overlapped by broad H₂O peak), 3.82 (s, OMe, partly overlapped by broad H₂O peak), 5.04 (s, 2H, CH₂O), 5.94 (br s, NH₂), 6.21 (br s, NH₂), 6.99 (d, J=8 Hz, 1H, 3′-H), 7.11 (d, J=2 Hz, 1H, 6′-H), 7.21 (dd, J=8 Hz, J=2 Hz, 1H, 4′-H), 7.28-7.45 (m, 3H, 5″- and 6″-H, pyrimidine 6-H), 7.54 (m, 2H, 2″ and 4″-H). Anal. (C₂₂H₂₀N₄O₄.AcOH.1.25H₂O) Calc. C, 59.19; H, 5.48; N, 11.50; Found C, 58.96; H, 5.21; N, 11.52.

### Example 13

### 6-[5-(2,4-Diamino-pyrimidin-5-ylmethyl)-2,3-dimethoxy-phenyl]-hex-5-ynoic acid (Compound 35)

2,4-Diamino-5-(3′-iodo-4′,5′-dimethoxy-benzyl)pyrimidine (51). A solution of sodium methoxide was prepared by dissolving 46 mg (2.0 mmol) of sodium metal in 3 mL of absolute methanol. The solvent was evaporated under reduced pressure, the residue was dissolved in 5 mL of dimethylsulfoxide, 1.40 g (0.01 mol) of 3-morpholinopropionitrile was added. The mixture was placed in a 100° C. oil bath, a solution of 2.92 g (0.01 mol) of 2,4-dimethoxy-6-iodobenzaldehyde in 5 mL of dimethylsulfoxide was added slowly, and heating was continued for 20 min. After cooling, the mixture was partitioned between ethyl acetate and water containing 200 mg of citric acid. The organic layer was evaporated, the residue, consisting of morpholinonitrile 50, was then dissolved in 20 mL of absolute ethanol, solid aniline hydrochloride was added, and the solution was refluxed for 20 min.

Separately, a solution of sodium ethoxide was prepared by dissolving 0.69 g (0.03 mol) of sodium metal in 50 mL of absolute ethanol. Then 1.43 g (0.015 mol) of guanidine hydrochloride was added, and the mixture was swirled for 5 min and combined with the solution of 50 prepared above. The mixture was refluxed for 6.5 h, then chilled to −30 ° C. until a copious solid formed, which was collected and stirred with 40 mL of water to remove sodium chloride. The washed solid was then freeze-dried to obtain 0.65 g of crude 51. Recrystallization from 95% EtOH gave 541 mg of off-white solid, mp 219-220° C.; NMR (DMSO-d₆) δ 1.01 (t, J=8 Hz, 2H, CH₃, ca. 0.67 EtOH of crystallization), 3.41 (p, J=8 Hz, <2H, CH₃, ca 0.67 EtOH of crystallization), 3.48 (s, 2H, bridge CH₂), 3.62 (s, 3H, MeO), 3.74 (s, 3H, MeO), 4.29 (t, J=5 Hz, OH of EtOH), 5.66 (br s, 2H, NH₂), 6.06 (br s, 2H, NH₂), 6.94 (s, 1H, phenyl), 7.10 (s, 1H, phenyl), 7.52 (s,1H, pyrimidine).

### 2,4-Diamino-5-[3′,4′-dimethoxy-5′-(5-carboxy-1-pentynyl)benzyl]-pyrimidine (49)

Step 1. A solution of 1.96 g (0.02 mol) of 5-pentynoic acid in 25 mL of dimethylformamide was stirred with 3.25 g (0.01 mol) cesium carbonate for 10 min, followed by addition of 3.42 g (2.38 mL0, 0.02 mol) of benzyl bromide. After 18 h at room temperature, the solvent was evaporated and the residue partitioned between ethyl acetate and water. Evaporation of the organic layer afforded 4.29 g of impure benzyl 5-pentynoate which was used directly in the next step (the NMR spectrum indicate that the impurity was unreacted benzyl bromide).

Step 2. A mixture of 240 mg (0.62 mmol) of 51 (dried at 75° C. in vacuo over phosphorous pentoxide) 202 mg (1.0 mmol) of benzyl 5-hexynoate from the preceding step, 10 mg of dichlorobis(triphenylphosphine)palladium (II) and 10 mg of bromotris(triphenylphosphine)copper(II) in 3 mL of dimethylformamide and 3 mL of triethylamine was heated at 65° C. for 18 h under nitrogen. The solvent was evaporated and the residue was swirled with isooctane, which was then decanted. The residue was dissolved in 3 mL of dimethylsulfoxide and treated dropwise with 2 mL of 2M sodium hydroxide. The solution was then diluted to 40 mL with water and a small amount of undissolved solid was filtered off. The filtrate was adjusted to pH 9.4 with 10% acetic acid. HPLC (Luna C₁₈ silica gel, 15% MeCN in 0.1 M NH₄OH, H 7.4, 1.0 m/min 280 nm) showed 49 as the major peak R_{f} 13 min. Preparative HPLC was performed with the same eluent system except that the acetonitrile concentration was gradually increased from 12% initially to 15% at the end. The pooled eluates were concentrated and freeze-dried. The residue was dissolved in dilute NaOH, filtered, and acidified with 10% acetic acid. Although the product did not precipitate immediately, a solid finally formed after 15 min when the solution was chilled. The mixture was frozen and thawed in order to improve the granularity of the precipitate, and the resulting solid was collected and freeze-dried, affording 146 mg (58%) of 49 as a white solid, mp 222-224° C. dec; IR (KBr) 3580, 3340, 3160, 2930, 2830w, 1680, 1655, 1575, 1505, 1485, 1460, 1420, 1400, 1335, 1265, 1230, 1180, 1140, 1120, 1030, 1000, 945, 805, 770, 690, 650 cm⁻¹; NMR (DMSO-d₆) δ 1.68 (p, J=7 Hz, 2H, C-3 CH₂), 1.84 (s, <1H, CH₃COOH), 2.39 (m overlapping DMSO, C-2 and C-4 CH₂), 3.49 (s overlapping H₂O, bridge CH₂), 3.64 (s, 3H, MeO), 3.69 (s, 3H, MeO), 5.66 (s, 2H, NH₂), 6.03 (s, 2H, NH₂), 6.03 (s, 1H, phenyl), 6.61 (s, 1H, phenyl), 7.48 (s, 1H, pyrimidine).

Anal: Calcd for C₁₉H₂₂N₄O₄0.25CH₃COOH.1.75H₂O: C, 56.17; H, 6.41; N, 13.44. Found: C, 56.22; H, 6.13; N, 13.40.

### Example 14

### Enzyme Assays and Data Analysis

Standardized spectrophotometric determination of Pc, Tg, Ma, and rat liver DHFR inhibition was performed as described previously. IC₅₀ values were obtained with the aid of the curve-fitting program Prism 3.0. The data were transformed to % of uninhibited control for enzyme activity (y-axis) and the log of the molar drug concentration (x-axis). The analyses for individual curves had degrees of freedom ranging from 6 (2 curves) to 42 (1 curve); the mode for degrees of freedom was 12 (14 curves). The r² value for individual-curves, determined using the program InStat 2.03, ranged from 0.71 (1 curve) to >0.99 (24 curves); all but two curves had r² values of >0.93. The range of SI values for each compound was calculated from the 95% confidence intervals for the individual IC₅₀ values for that compound; all were referenced to rat liver DHFR. As an example of the reproducibility of these methods, when lb was assayed against Pc DHFR on three independent occasions, the mean ±SEM (standard error of the mean) was found to be 0.054±0.0023 μM (i.e., the SEM was 4.3% of the mean). In quality control assays using TMP against the same enzyme, the SEM was 6.3% of the mean.

1. A compound according to Formula 1: 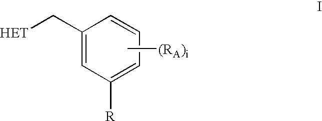
wherein
HET is selected from 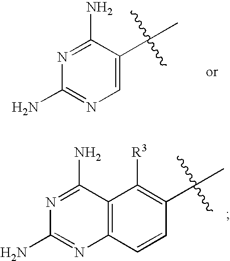
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 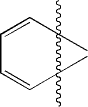
which may be optionally substituted;
R is L-(CR¹R²)ₙ—C(O)—X or a group of the formula: 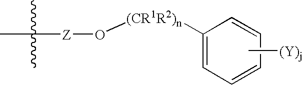
L is a single bond, an optionally substituted 1,2-vinylidene group, or a 1,2 acetylene-diyl group;
X is selected from OH, SH, NH₂, optionally substituted alkoxy, optionally substituted benzyloxy, or optionally substituted aryloxy and salts thereof; and
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
i is an integer from 0 to about 4;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

2. The compound of claim 1 which is a compound according to Formula II: 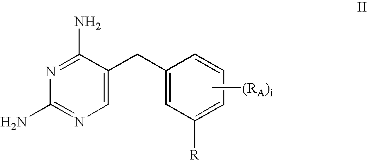
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 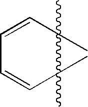
which may be optionally substituted;
R is L-(CR¹R²)ₙ—C(O)—X or a group of the formula: 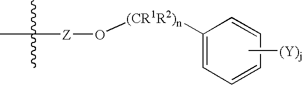
L is a single bond, an optionally substituted 1,2-vinylidene group, or a 1,2 acetylene-diyl group;
X is selected from OH, SH, NH₂, optionally substituted alkoxy, optionally substituted benzyloxy, or optionally substituted aryloxy and salts thereof; and
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
i is an integer from 0 to about 4;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

3. The compound of claim 1 which is a compound according to Formula III: 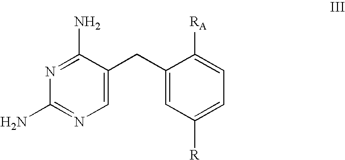
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
R is L-(CR¹R²)ₙ—C(O)—X or a group of the formula: 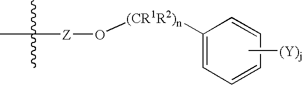
L is a single bond, an optionally substituted 1,2-vinylidene group, or a 1,2 acetylene-diyl group;
X is selected from OH, SH, NH₂, optionally substituted alkoxy, optionally substituted benzyloxy, or optionally substituted aryloxy and salts thereof; and
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

4. A compound according to claim 1, which is according to Formula IV:

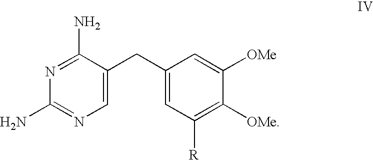

5. The compound of claim 1 which is a compound according to Formula V: 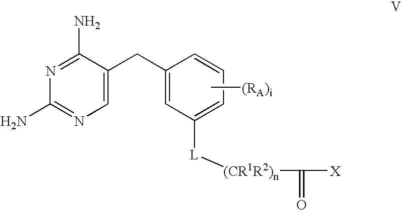
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 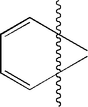
which may be optionally substituted;
L is a single bond or a 1,2 acetylene-diyl group;
X is selected from OH, SH, NH₂, optionally substituted alkoxy, optionally substituted benzyloxy, or optionally substituted aryloxy and salts thereof; and
n is an integer of from 1 to about 12; or
i is an integer from 0 to about 4;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

6. The compound of claim 1 which is a compound according to Formula VI: 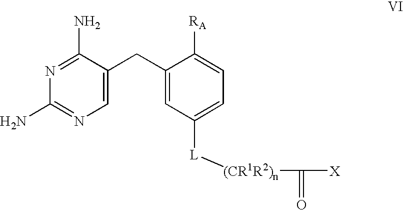
wherein
L is a single bond or a 1,2 acetylene-diyl group;
X is selected from OH, SH, NH₂, optionally substituted alkoxy, optionally substituted benzyloxy, or optionally substituted aryloxy and salts thereof; and
n is an integer of from 1 to about 12; or
j is an integer from 0 to about 5; and
R_{A}, R¹, and R² are independently selected at each occurrence from the group consisting of hydrogen, methyl, ethyl, chloro, fluoro, hydroxy, amino, trifluoromethoxy, or methoxy.

7. A compound of claim 3, wherein L is a single bond.

8. A compound of claim 3, wherein L is a 1,2-acetylenediyl group.

9. A compound of claim 6, wherein the compound is according to the formula: 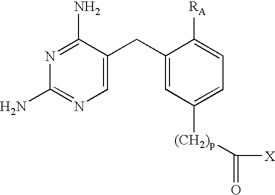
wherein p is an integer from 3 to about 14.

10. A compound of claim 6, wherein the compound is according to the formula: 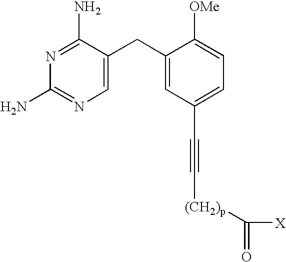
wherein p is an integer from 1 to about 12.

11. The compound of claim 1 which is a compound according to Formula VII: 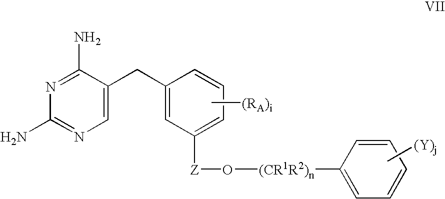
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 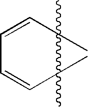
which may be optionally substituted;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
i is an integer from 0 to about 4;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

12. The compound of claim 1 which is a compound according to Formula VIII: 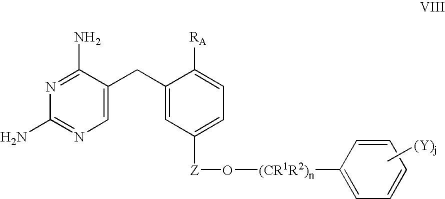
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹ and R²are independently selected at each occurrence from the group consisting of hydrogen, methyl, ethyl, chloro, fluoro, hydroxy, amino, trifluoromethoxy, or methoxy.

13. A compound of claim 12, wherein the compound is according to Formula IX: 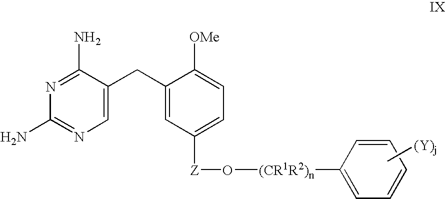
wherein
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

14. A compound of claim 12, wherein the compound is according to formula: 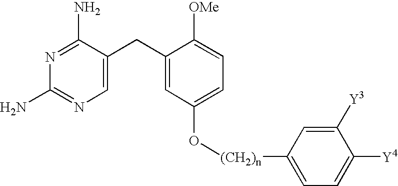
wherein at least one Y³ and Y⁴ is a carboxylic acid residue or a salt thereof.

15. The compound of claim 1 which is a compound according to Formula X: 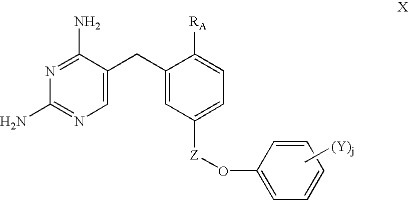
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a hydrocarbon chain having from 1 to about 6 carbon atoms in the chain and between 0 and about 2 double or triple bonds in the chain;
j is an integer from 0 to about 5; and
R¹ and R² are independently selected at each occurrence from the group consisting of hydrogen, methyl, ethyl, chloro, fluoro, hydroxy, amino, trifluoromethoxy, or methoxy.

16. A compound of claim 15, wherein the compound is according to the formula 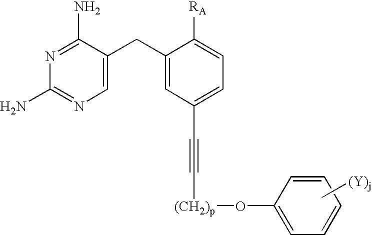
wherein
at least one Y³ and Y⁴ is a carboxylic acid residue or a salt thereof; and
p is an integer of from 1 to about 4.

17. A compound of claim 15, wherein the compound is according to the formula 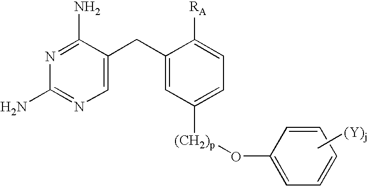
wherein
at least one Y³ and Y⁴ is a carboxylic acid residue or a salt thereof; and
p is an integer of from 3 to about 6.

18. The compound of claim 1 which is a compound according to Formula XI: 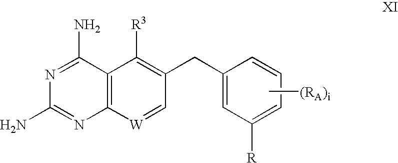
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 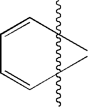
which may be optionally substituted;
R is L-(CR¹R²)ₙ—C(O)—X or a group of the formula: 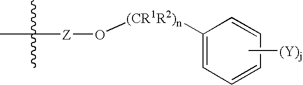
L is a single bond, an optionally substituted 1,2-vinylidene group, or a 1,2 acetylene-diyl group;
W is a CH group or N;
X is selected from OH, SH, NH₂, optionally substituted alkoxy, optionally substituted benzyloxy, or optionally substituted aryloxy and salts thereof; and
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
i is an integer from 0 to about 4;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

19. The compound of claim 1 which is a compound according to Formula XII: 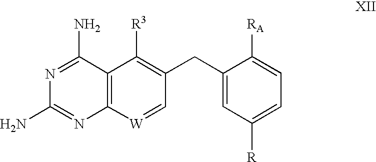
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
R is L-(CR¹R²)ₙ—C(O)—X or a group of the formula: 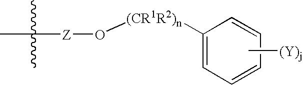
L is a single bond, an optionally substituted 1,2-vinylidene group, or a 1,2 acetylene-diyl group;
W is a CH group or N;
X is selected from OH, SH, NH₂, optionally substituted alkoxy, optionally substituted benzyloxy, or optionally substituted aryloxy and salts thereof; and
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

20. A compound according to claim 19, which is according to Formula XIII:

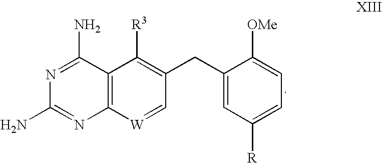

21. The compound of claim 1 which is a compound according to Formula XIV: 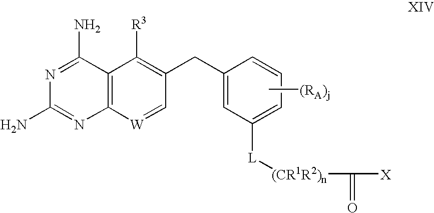
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 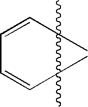
which may be optionally substituted;
L is a single bond or a 1,2 acetylene-diyl group;
W is a CH group or N;
X is selected from OH, SH, NH₂, optionally substituted alkoxy, optionally substituted benzyloxy, or optionally substituted aryloxy and salts thereof; and
n is an integer of from 1 to about 12; or
i is an integer from 0 to about 4;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

22. The compound of claim 1 which is a compound according to Formula XV: 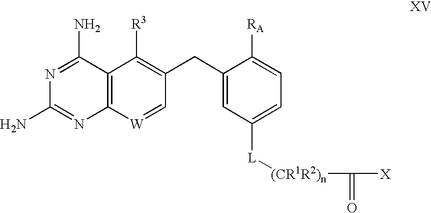
wherein
L is a single bond or a 1,2 acetylene-diyl group;
W is a CH group or N;
X is selected from OH, SH, NH₂, optionally substituted alkoxy, optionally substituted benzyloxy, or optionally substituted aryloxy and salts thereof; and
n is an integer of from 1 to about 12; or
j is an integer from 0 to about 5; and
R_{A}, R¹, R², R³ are independently selected at each occurrence from the group consisting of hydrogen, methyl, ethyl, chloro, fluoro, hydroxy, amino, trifluoromethoxy, or methoxy.

23. A compound of claim 19, wherein L is a single bond.

24. A compound of claim 19, wherein L is a 1,2-acetylenediyl group.

25. A compound of claim 22, wherein the compound is according to the formula: 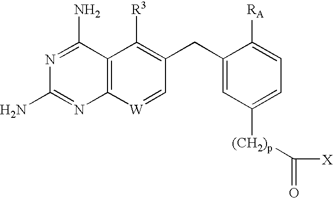
wherein p is an integer from 3 to about 14.

26. A compound of claim 22, wherein the compound is according to the formula: 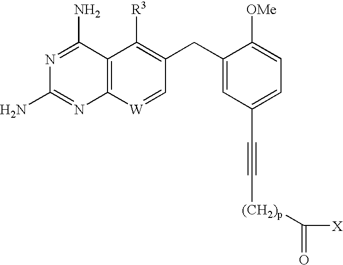
wherein p is an integer from 1 to about 12.

27. The compound of claim 1 which is a compound according to Formula XVI: 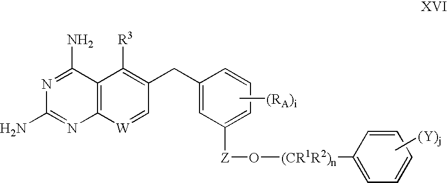
wherein
R_{A} is independently selected at each occurrence of R_{A} from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro; or
two adjacent R_{A} groups taken in combination form a group of the formula: 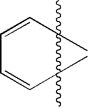
which may be optionally substituted;
W is a CH group or N;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
i is an integer from 0 to about 4;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

28. The compound of claim 1 which is a compound according to Formula XVII: 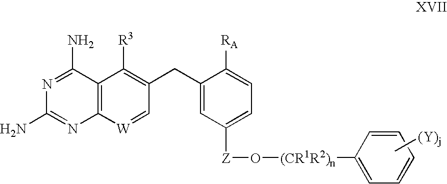
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
W is a CH group or N;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

29. A compound of claim 28, wherein the compound is according to Formula XVIII: 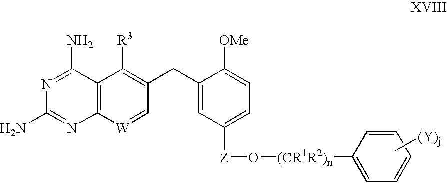
wherein
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a single bond or hydrocarbon chain having from 1 to about 6 carbon atoms in the chain and between 0 and about 2 double or triple bonds in the chain;
n is an integer of from 0 to about 12, wherein the sum of n and the number of carbon atoms in the hydrocarbon chain of the Z group is greater than 0;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

30. A compound of claim 29 wherein the compound is according to formula: 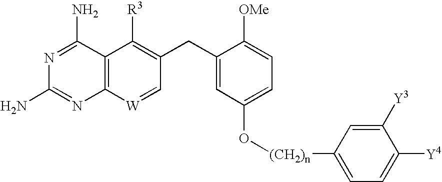
wherein at least one Y³ and Y⁴ is a carboxylic acid residue or a salt thereof.

31. The compound of claim 1 which is a compound according to Formula XIX: 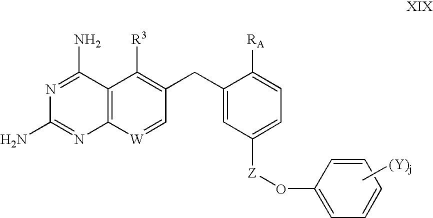
wherein
R_{A} is selected from the group consisting of hydrogen, optionally substituted alkyl, optionally substituted alkenyl, optionally substituted alkynyl, optionally substituted alkoxy, optionally substituted haloalkyl, chloro, fluoro, hydroxy, amino, optionally substituted mono and di-alkylamino, and nitro;
W is a CH group or N;
Y is independently selected at each occurrence from the group consisting of —C(O)—X, hydrogen, methyl, ethyl, chloro, fluoro, hydroxy, amino, trifluoromethoxy, or methoxy, wherein at least one occurrence of Y is CO₂H or a salt thereof;
Z is a hydrocarbon chain having from 1 to about 6 carbon atoms in the chain and between 0 and about 2 double or triple bonds in the chain;
j is an integer from 0 to about 5; and
R¹, R², R³ are independently selected at each occurrence from the group consisting of hydrogen, methyl, ethyl, chloro, fluoro, hydroxy, amino, trifluoromethoxy, or methoxy.

32. A compound of claim 31, wherein the compound is according to the formula 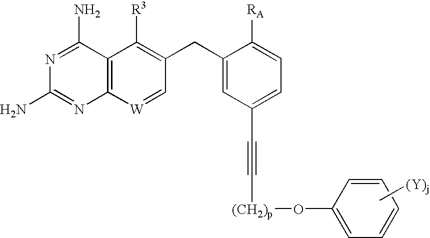
wherein
at least one Y³ and Y⁴ is a carboxylic acid residue or a salt thereof; and
p is an integer of from 1 to about 4.

33. A compound of claim 31, wherein the compound is according to the formula 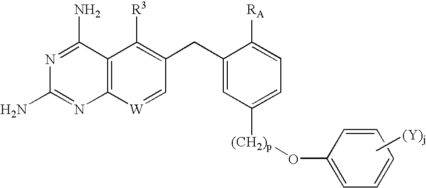
wherein
at least one Y³ and Y⁴ is a carboxylic acid residue or a salt thereof; and
p is an integer of from 3 to about 6.

34. A compound according to claim 2 of the formula: 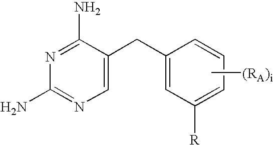
wherein
R_{A} is independently selected at each occurrence of R_{A} from the group consisting of hydrogen, C₁₋₆alkyl, C₂₋₆alkenyl, C₂₋₆alkynyl, C₁₋₆alkoxy, C₁₋₆haloalkyl, chloro, fluror, hydroxy, amino, optionally substituted mono and di-alkylamino, and nitro;
R is L-(CR¹R²)ₙ—C(O)—X;
L is a single bond, an optionally substituted 1,2-vinylidene group, or a 1,2 acetylene-diyl group;
X is selected from OH, SH, NH₂, optionally substituted alkoxy, optionally substituted benzyloxy, or optionally substituted aryloxy and salts thereof; and
n is an integer of from 0 to about 12;
i is an integer from 0 to about 4; and
R¹ and R² are independently selected at each occurrence from the group consisting of hydrogen, methyl, ethyl, chloro, fluoro, hydroxy, amino, trifluoromethoxy, or methoxy.

35. A compound according to claim 2 of the formula: 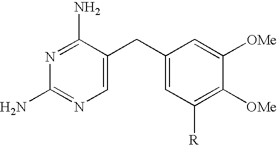
wherein
L is a single bond, an optionally substituted 1,2-vinylidene group, or a 1,2 acetylene-diyl group;
X is selected from OH, SH, NH₂, optionally substituted alkoxy, optionally substituted benzyloxy, or optionally substituted aryloxy and salts thereof; and
n is an integer of from 0 to about 12;
R¹ and R² are independently selected at each occurrence from the group consisting of hydrogen, methyl, ethyl, chloro, fluoro, hydroxy, amino, trifluoromethoxy, or methoxy.

36. A compound according to claim 34, wherein L is a 1,2-acetylene-diyl group.

37. A compound of claim 34, wherein X is OH or C₁₋₆alkoxy.

38. A compound of claim 34, wherein n is an integer of from 1 to 10.

39. A compound of claim 38, wherein n is an integer of from 2 to 8.

40. A compound of claim 34, wherein n is 3.

41. A compound according to claim 34, wherein the compound is represented by the formula: 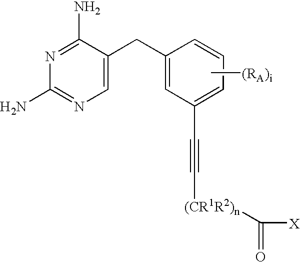
wherein
i is 1 or 2;
n is an integer of from 1 to about 10;
R¹ and R² are independently selected at each occurrence from the group consisting of hydrogen, methyl, ethyl, hydroxy, and methoxy; and
R_{A} is independently selected at each occurrence of R_{A} from the group consisting of hydrogen, C₁₋₆alkyl, and C₁₋₆alkoxy.

42. A compound according to claim 34, wherein the compound is represented by the formula: 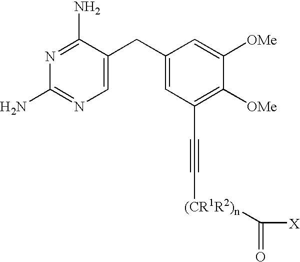
wherein
n is an integer of from 1 to about 10; and
R¹ and R are independently selected at each occurrence from the group consisting of hydrogen, methyl, ethyl, hydroxy, and methoxy.

43. A compound of claim 42, wherein n is an integer of from 2-4; X is OH; and each occurrence of R¹ is hydrogen and R² is independently selected at each occurrence from hydrogen, methyl, ethyl.

44. A compound of claim 1, wherein the compound is selected from the group consisting of:
2,4-Diamino-5-[5′-(4-carboxy-1-butynyl)-2′-methoxy)benzyl]pyrimidine;
2,4-Diamino-5-[5′-(4-carboxy-1-pentynyl)-2′-methoxy)benzyl]pyrimidine;
2,4-Diamino-5-[5′-(6-carboxy-1-hexynyl )-2′-methoxy)benzyl]pyrimidine;
2,4-Diamino-5-[5′-(4-carboxybutyl)-2′-methoxy)benzyl]pyrimidine;
2,4-Diamino-5-[5′-(5-carboxypentyl)-2′-methoxy)benzyl]pyrimidine;
2,4-Diamino-5-[5′-(6-carboxyhexyl)-2′-methoxy)benzyl]pyrimidine;
2,4-Diamino-5-[2′-methoxy-5′-(3″-carboxybenzyloxy)benzylpyrimidine;
2,4-Diamino-5-(2′-methoxy-5′-(4″-carboxybenzyloxy)benzylpyrimidine;
2,4-Diamino-5-[2′-methoxy-5′-[3-(2″-carboxyphenoxy)propyn-1-yl]benzyl]pyrimidine;
6-[5-(2,4-Diamino-pyrimidin-5-ylmethyl)-2,3-dimethoxy-phenyl]-hex-5-ynoic acid; and
2,4-Diamino-5-[2′-methoxy-5′-[3-(3″-carboxyphenoxy)-1-propynyl]benzyl]pyrimidine.

45-48. (canceled)

49. A compound of claim 1, wherein the compound has an IC₅₀ of 1 μM or less against a DHFR enzyme.

50-53. (canceled)

54. A compound of claim 49 wherein the DHFR enzyme is a DHFR enzyme of organism causing a disease or infection selected from malaria, trypanosomiasis, leprosy, toxoplasmosis, and pneumocvstis carinii pneumonia.

55-56. (canceled)

57. A compound of claim 54, wherein the parasite organism is selected from the group consisting of Pneumocystis carinii, Toxoplasma gondii, and Mycobacterium avium.

58. A compound according to claim 1, wherein the binding selectivity of the compound, e.g., the ratio of the compound's IC₅₀ for binding mammalian DHFR enzymes to the compound's IC₅₀ for binding IC₅₀ for binding parasitic DHFR enzymes, is greater than about 1.

59-65. (canceled)

66. A compound according to claim 1, wherein the compound is a lipophilic inhibitor of dihydrofolate reductase.

67. A pharmaceutical composition comprising a compound of claim 1 and a pharmaceutically acceptable carrier.

68. A method for treating a mammal suffering or susceptible to a parasitic infection or disorder, comprising administering to the mammal an effective amount of a compound or composition of claim 1.

69. A method of claim 68 wherein the mammal is immuno-compromised.

70. The method of claim 68, wherein the mammal is HIV-positive.

71. The method of claim 68 wherein the mammal is suffering from an acquired immune deficiency disorder.

72. The method of claim 68, wherein the mammal is suffering from an autoimmune disorder or disease.

73. The method of claim 68, wherein the mammal has a parasitic infection.

74. The method of claim 73, wherein the parasitic infection is a Pneumocystis carini (Pc), Toxoplasma gondii (Tg), or a Mycobacterium avium (Ma) infection.

75. A method for treating an immuno-compromised mammal comprising administering to the mammal an effective amount of a compound or composition of claim 1.

76. The method of claim 75, wherein the mammal is HIV-positive.

77. The method of claim 75, wherein the mammal has AIDS.

78. The method of claim 75, wherein the mammal has an autoimmune disorder.

79. The method of claim 68 wherein the mammal is a human.

80. The method of claim 75 wherein the mammal is a human.
